(19) United States
(12) Patent Application Publication
(10) Pub. No.: US 2025/0255675 A1
(43) Pub. Date: Aug. 14, 2025

(54) Extended Reality Systems And Methods For Surgical Applications

(71) Applicant: Stryker Corporation, Portage, MI (US)

(72) Inventors: Thang Sy, Sacramento, CA (US); Steven D. Scherf, Oakland, CA (US); Mayank Kumar, San Jose, CA (US); Siddarth Satish, Portola Valley, CA (US); Sandesh Basnet, Menlo Park, CA (US); Matthew Carter, Auckland (NZ)

(73) Assignee: Stryker Corporation, Portage, MI (US)

(21) Appl. No.: 19/046,592

(22) Filed: Feb. 6, 2025

Related U.S. Application Data
(60) Provisional application No. 63/551,719, filed on Feb. 9, 2024.

(51) Int. Cl. A61B 34/20; A61B 34/10; A61B 90/00

(52) U.S. Cl. CPC A61B 34/20; A61B 34/10; A61B 90/361; A61B 2034/102; A61B 2034/105; A61B 2034/107; A61B 2090/365

(57) ABSTRACT

Extended reality systems and methods for use in a surgical procedure involve a head-mounted device (HMD) comprising an HMD display positionable in front of a user's eyes and a sensing system to sense control inputs of the user. Controller(s) coupled to the HMD receive control inputs from the sensing system to establish a pose of a view coordinate system in which to present a virtual object (e.g., a virtual information panel) related to the surgical procedure. The pose can be established using a virtual alignment guide. The controller(s) define the view coordinate system relative to a world coordinate system after the pose of the view coordinate system is established. The controller(s) recognize surgical information, and in response, automatically present the virtual object on the HMD display combined with a real-world view and at a predetermined position and orientation within the view coordinate system.

### CROSS-REFERENCE TO RELATED APPLICATIONS

The subject application claims priority to and all the benefits of U.S. provisional patent application No. 63/551,719, filed Feb. 9, 2024, the entire contents of which are hereby incorporated by reference.

### BACKGROUND

Extended reality (xR) is playing an increasingly key role in aiding surgical procedures. By wearing an xR headset, surgeons can perform procedures while the headset displays xR graphics related to the surgery. The xR graphics are presented directly in-line with the surgeon's view of the surgical site.

While there have been significant developments in surgical xR, conventional xR systems have several shortcomings. For example, many surgeons are accustomed to traditional (manual) means of performing surgery and are reluctant to utilize xR systems due to their complexity. When performing a surgery, surgeons want to know exactly where to find information, when needed. In many conventional surgical systems, a monitor is provided in the operating room to provide surgically relevant information. This monitor is often attached to a movable cart, e.g., of a surgical navigation system, and positioned near the surgical site. Surgeons know that they can find the surgically relevant information on the monitor.

However, in conventional xR systems, the placement of xR graphics related to the surgery is not so predictable. Conventional xR systems do not adequately provide the user with intuitive or easily controllable means for customizing placement of the xR graphics provided by the xR headset. Many times, the xR system may display such xR graphics in a “one-size-fits-all” location or require the user to manually move the location of each xR graphic, one-by-one. Consequently, the attention needed to place xR graphics may cause significant disruption or distraction to the surgeon or the surgical procedure. Additionally, surgeons may want certain information to be displayed at appropriate times. Conventional xR systems often fail to discriminate when certain information is displayed or how much information is displayed. In turn, such xR systems typically display information at improper times and can display an overwhelming amount of information, which can impair the view and attention of the surgeon, thereby amplifying the disruption or distraction.

Additionally, conventional xR systems lack the “plug and play” compatibility that surgeons and healthcare facilities demand. Conventional xR systems are often not well-suited to be seamlessly compatible with existing surgical systems without significant re-development and re-design of the extended reality system and/or the surgical system. There are countless manufacturers of xR systems and surgical systems. However, unless the systems were specifically developed to work together, the xR system typically will not be adapted to function in conjunction with the surgical system, and vice-versa. Healthcare facilities or surgeons may invest significant resources in purchasing xR headsets, only to discover that the headsets may not be compatible with the broad range of surgical systems and software required for various surgical procedures.

Furthermore, existing means of processing and communicating a video data to an xR headset suffer from various issues, such as latency, lack of efficient data transfer, and packet loss with wireless signal interference. These issues may be tolerable for certain uses, such as personal use. However, for surgical applications, the processing and communicating of the video stream to the xR headset should be fast and robust. As such, there is a need to provide technical solutions to address the various issues described above.

### SUMMARY

This Summary introduces a selection of concepts in a simplified form that are further described below in the Detailed Description below. This Summary is not intended to limit the scope of the claimed subject matter nor identify key features or essential features of the claimed subject matter.

According to a first aspect, an extended reality system for use in a surgical procedure, comprising: a head-mounted device (HMD) comprising an HMD display positionable in front of a user's eyes and a sensing system configured to sense control inputs of the user; and one or more controllers coupled to the HMD and being configured to: receive control inputs from the sensing system to establish a pose of a view coordinate system in which to present a virtual object related to the surgical procedure; define and/or fix the view coordinate system relative to a world coordinate system after the pose of the view coordinate system is established; recognize surgical information; and in response to recognition of the surgical information, automatically present the virtual object on the HMD display combined with a real-world view and at a predetermined position and orientation within the view coordinate system.

Also provided are: a method of operating the extended reality system of the first aspect; a non-transitory computer-readable medium comprising instructions, which when executed by one or more processors, operate the extended reality system of the first aspect; a surgical system including the extended reality system of the first aspect; and a method of operating the surgical system including the extended reality system of the first aspect.

According to a second aspect, extended reality system is provided for use with a surgical navigation system that presents, on a display, a clinical application to provide guidance for a surgical procedure, the extended reality system comprising: a head-mounted device (HMD) comprising an HMD display positioned in front of a user's eyes and configured to combine a computer-generated graphic with a real-world view; and one or more controllers coupled to the HMD and being configured to: receive, from the surgical navigation system, a video stream of the clinical application; recognize surgical information from the video stream of the clinical application; and in response to recognition of the surgical information, automatically present, on the HMD display, a virtual object related to the surgical information and combined with the real-world view.

Also provided are: a method of operating the extended reality system of the second aspect; and a non-transitory computer-readable medium comprising instructions, which when executed by one or more processors, operate the extended reality system of the second aspect.

According to a third aspect, a surgical system is provided comprising: a head-mounted device (HMD) comprising an HMD display positioned in front of a user's eyes and configured to combine a computer-generated graphic with a real-world view; a surgical navigation system comprising a display, wherein the display of the surgical navigation system is spatially separated from the HMD, and wherein the surgical navigation system is configured to present, on the display, a clinical application configured to provide guidance for a surgical procedure; and one or more controllers coupled to the HMD and the surgical navigation system and being configured to: receive, from the surgical navigation system, a video stream of the clinical application; recognize surgical information from the video stream of the clinical application; and in response to recognition of the surgical information, automatically present, on the HMD display, a virtual object related to the surgical information and combined with the real-world view.

Also provided are: a method of operating the surgical system of the third aspect; and a non-transitory computer-readable medium comprising instructions, which when executed by one or more processors, operate the surgical system of the third aspect.

According to a fourth aspect, a connectivity system is provided that is configured to establish connectivity between an extended reality head-mounted device (HMD) and a separate host system/device that is configured to present a software application on a display, wherein the connectivity system comprises: a computing system; an input device coupled to the computing system and configured to receive a video stream of the software application from the host system/device; and an output device coupled to the computing system and configured to communicate with the HMD; wherein the computing system is configured to automatically: recognize information from the video stream of the software application; in response to recognition of the information, generate a virtual object related to the information; and communicate with the HMD to cause the HMD to present, on a display of the HMD, the virtual object combined with the real-world view.

Also provided are: a method of operating the connectivity system of the fourth aspect; a non-transitory computer-readable medium comprising instructions, which when executed by one or more processors, operate the connectivity system of the fourth aspect; a host system/device including the connectivity system of the fourth aspect; a method of operating the host system/device including the connectivity system the fourth aspect; an extended reality system including the connectivity system of the fourth aspect; a method of operating the extended reality system including the connectivity system the fourth aspect; an HMD including the connectivity system of the fourth aspect; a method of operating the HMD including the connectivity system the fourth aspect.

According to a fifth aspect, an extended reality system is provided, comprising: a head-mounted device (HMD) comprising an HMD display positionable in front of a user's eyes and a sensing system configured to sense control inputs of the user; and one or more controllers coupled to the HMD and being configured to: present, on the HMD display, an alignment guide configured to assist the user to define a position and an orientation of a view coordinate system in which to present one or more virtual objects, wherein the alignment guide is computer-generated and combined with a real-world view on the HMD display, and wherein the alignment guide comprises a position guide object dedicated to establishing the position for the view coordinate system and an orientation guide object dedicated to establishing the orientation for the view coordinate system; receive control inputs from the sensing system to enable selection and translational movement of the position guide object to establish the position of the view coordinate system; and receive control inputs from the sensing system to enable selection and rotational movement of the orientation guide object to establish the orientation of the view coordinate system.

Also provided are: a method of operating the extended reality system of the fifth aspect; a non-transitory computer-readable medium comprising instructions, which when executed by one or more processors, operate the extended reality system of the fifth aspect; a surgical system including the extended reality system of the fifth aspect; and a method of operating the surgical system including the extended reality system of the fifth aspect.

According to a sixth aspect, an extended reality system is provided, comprising: a head-mounted device (HMD) comprising an HMD display positionable in front of a user's eyes and a sensing system configured to sense control inputs of the user; and one or more controllers coupled to the HMD and being configured to: present, on the HMD display, an alignment guide configured to assist the user to define a position and an orientation of a virtual object, wherein the alignment guide is computer-generated and combined with a real-world view on the HMD display, and wherein the alignment guide comprises a position guide object dedicated to establishing the position for the virtual object and an orientation guide object dedicated to establishing the orientation for the virtual object; receive control inputs from the sensing system to enable selection and translational movement of the position guide object to establish the position for the virtual object; and receive control inputs from the sensing system to enable selection and rotational movement of the orientation guide object to establish the orientation of the virtual object.

Also provided are: a method of operating the extended reality system of the sixth aspect; a non-transitory computer-readable medium comprising instructions, which when executed by one or more processors, operate the extended reality system of the sixth aspect; a surgical system including the extended reality system of the sixth aspect; and a method of operating the surgical system including the extended reality system of the sixth aspect.

According to a seventh aspect, an extended reality system is provided, comprising: a head-mounted device (HMD) comprising an HMD display positionable in front of a user's eyes and a sensing system configured to sense control inputs of the user; and one or more controllers coupled to the HMD and being configured to: present, on the HMD display, an alignment guide object configured to assist the user to define a position and an orientation of a virtual object, wherein the alignment guide object is computer-generated and combined with a real-world view on the HMD display, and wherein the alignment guide object is spatially separate and distinct from the virtual object; receive control inputs from the sensing system to enable translational movement of the alignment guide object to establish the position for the virtual object; and receive control inputs from the sensing system to enable rotational movement of the alignment guide object to establish the orientation of the virtual object.

Also provided are: a method of operating the extended reality system of the seventh aspect; a non-transitory computer-readable medium comprising instructions, which when executed by one or more processors, operate the extended reality system of the seventh aspect; a surgical system including the extended reality system of the seventh aspect; and a method of operating the surgical system including the extended reality system of the seventh aspect.

According to an eighth aspect, a surgical system is provided, comprising: a head-mounted device (HMD) comprising an HMD display positionable in front of a user's eyes; a camera source; one or more controllers coupled to the HMD and the camera source, and being configured to: recognize surgical information from the camera source; and in response to recognition of the surgical information, automatically present a virtual object on the HMD display combined with a real-world view and at a predetermined pose, wherein the virtual object and the predetermined pose are based on the recognized surgical information.

Also provided are: a method of operating the surgical system of the eighth aspect; a non-transitory computer-readable medium comprising instructions, which when executed by one or more processors, operate the surgical system of the eighth aspect; an HMD of the eighth aspect; and a method of operating the HMD including of the eighth aspect.

According to a ninth aspect, a connectivity system is provided that is configured to establish connectivity between an extended reality head-mounted device (HMD) and a separate host system/device that is configured to present a software application on a display, wherein the connectivity system comprises: a computing system; an input device coupled to the computing system and configured to receive video data of the software application from the host system/device; and an output device coupled to the computing system and configured to wirelessly communicate with the HMD; wherein the computing system is configured to: receive the video data of the software application; model the video data into a custom data type; and wirelessly communicate the modeled video data to the HMD.

Also provided are: a method of operating the connectivity system of the ninth aspect; a non-transitory computer-readable medium comprising instructions, which when executed by one or more processors, operate the connectivity system of the ninth aspect; a host system/device including the connectivity system of the ninth aspect; a method of operating the host system/device including the connectivity system the ninth aspect; an extended reality system including the connectivity system of the ninth aspect; a method of operating the extended reality system including the connectivity system the ninth aspect; an HMD including the connectivity system of the ninth aspect; a method of operating the HMD including the connectivity system the ninth aspect.

According to a tenth aspect, an extended reality system is provided for use with a separate host system/device that presents, on a display, a software application, the extended reality system comprising: a head-mounted device (HMD) comprising an HMD display positioned in front of a user's eyes and configured to combine a computer-generated graphic with a real-world view; and one or more controllers coupled to the HMD and being configured to: receive, from the host system/device, a video stream of the software application; recognize information from the video stream of the software application; and in response to recognition of the information, automatically present, on the HMD display, a virtual object related to the information and combined with the real-world view.

Also provided are: a non-transitory computer-readable medium comprising instructions, which when executed by one or more processors, operate the extended reality system of the tenth aspect; a method of operating the extended reality system of the tenth aspect; an HMD of the tenth aspect; and a method of operating the HMD of the tenth aspect.

Any of the above aspects may be combined, in whole or in part.

Any of the above aspects may be combined with any of the following implementations. Any of the following implementations may be utilized in part, or in whole, with any of the above aspects. The implementations are:

The surgical procedure may involve a target site. The target site may be an anatomical joint, such as a knee, hip, shoulder, ankle, or spine. The controller(s) can receive control inputs from a sensing system to establish a position of the view coordinate system to be located directly above or adjacent to the target site. To establish the pose of the view coordinate system, the controller(s) can present, on the HMD display, an alignment guide to assist the user to define the pose of the view coordinate system. The alignment guide can be computer-generated and combined with the real-world view on the HMD display. The alignment guide can be spatially separate and distinct from the virtual object and not presented at the same time as the virtual object. The alignment guide can include a position guide object dedicated to establishing a position for the view coordinate system. The alignment guide can include an orientation guide object dedicated to establishing an orientation for the view coordinate system. The view coordinate system can comprise an origin. The position guide object can be dedicated to establishing the position of the origin of the view coordinate system. The orientation guide object can be dedicated to establishing the orientation of the view coordinate system defined relative to the origin. The position guide object can be a first volumetric object. The orientation guide object can be a second volumetric object. The controller(s) can present the position guide object as being spaced apart from the orientation guide object. The first volumetric object and/or the second volumetric object can be a ball. The controller(s) can present the first volumetric object with a first color and/or present the second volumetric object with a second color different from the first color. A straight object can be virtually coupled between the position guide object and the orientation guide object. The straight object can be rigidly fixed to both the position guide object and the orientation guide object such that the straight object, the position guide object, and the orientation guide object collectively form a virtual rigid body. The straight object can have a fixed length such that the position guide object and the orientation guide object are spatially constrained relative to one another by the fixed length of the straight object. The straight object can have a variable length. The alignment guide object can alternatively be a 3D object comprising a directional feature to indicate orientation. The 3D object can be a sphere and the directional feature can be a virtual indicia on a surface of the sphere, or a straight object extending from the sphere. The 3D object can be any other 3D shape.

The controller(s) can receive control inputs from the sensing system to enable selection and translational movement of the position guide object to establish the position of the view coordinate system. The controller(s) can receive control inputs from the sensing system to enable selection and rotational movement of the orientation guide object to establish the orientation of the view coordinate system. The real-world can include a world coordinate system. The view coordinate system can be defined and/or fixed relative to the world coordinate system after the position and the orientation of the view coordinate system are established. The controller(s) can save, in a non-transitory memory, the position and the orientation of the view coordinate system established by the user. The controller(s) can retrieve from the non-transitory memory the established position, and the orientation of the view coordinate system during a subsequent use of the HMD by the user. The controller(s) can request establishment of the position of the view coordinate system with the position guide object prior to establishment of the orientation of the view coordinate system with the orientation guide object. The control inputs can include a gaze input. The controller(s) can utilize the gaze input to enable selection of the position guide object and/or enable selection of the orientation guide object. The gaze in can be a look and stare input or dwell time input. The control inputs can include a hand gesture input. The controller(s) can utilize the hand gesture inputs to enable translational movement of the position guide object and/or enable rotational movement of the orientation guide object. The hand gesture input can be a finger pinch gesture that grasps a virtual object or that is located away from the virtual object. The controller(s) can simultaneously present the virtual object and the alignment guide on the HMD display. The controller(s) can translate the virtual object in correspondence with translational movement of the position guide object. The controller(s) can rotate the virtual object in correspondence with rotational movement of the orientation guide object.

The one or more virtual objects can be computer-generated. The controller(s) can present, on the HMD display, the one or more virtual objects within the view coordinate system and combined with the real-world view. The virtual object can be related to the surgical information. The virtual object can be one or more virtual objects. The virtual object can be a virtual information panel. The virtual information panel can display information related to the surgical information. The virtual object can be a 3D surgical object including one or more of: a 3D model of a bone, a 3D model of an implant, and a 3D surgical plan.

The controller(s) can receive, from a surgical navigation system, a video stream of a software application or clinical application. The software application or clinical application can be presented on a display of a host system/device or surgical navigation system. The software application or clinical application can be presented on the display of the host system/device or surgical navigation system concurrently while the virtual object is presented on the HMD display. The software application or clinical application may not be presented at all on the display of the host system/device or surgical navigation system. The controller(s) can recognize the information from the video stream of the software application or clinical application. The information can be surgical information. In response to recognition of the information, the controller(s) can automatically present the virtual object on the HMD display. The controller(s) can recognize the information from the video stream of the software application or clinical application by automatically identifying text and/or imagery presented by the software application or clinical application. The software application or clinical application can have a plurality of different screens, e.g., related to the surgical procedure. Each screen can have a unique identification, such as a title, or information on the screen to identify its contents or context. The controller(s) can recognize the information from the video stream of the software application or clinical application by automatically identifying the text and/or imagery of the identification of one of the screens. The information can be a step of the surgical procedure. The step of the surgical procedure can be one of: a pre-operative planning step, an operating room setup step, an anatomical registration step, an intra-operative planning step, an anatomical preparation step, or a post-operative evaluation step. In response to recognition of the information, the controller(s) can automatically present the one or more virtual objects on the HMD display at a predetermined position and orientation within the view coordinate system, wherein the one or more virtual objects relate to the information. The predetermined position and orientation of the one or more virtual objects within the view coordinate system can be based on: a default position and orientation; or a user-defined position and orientation. The controller(s) can duplicate a portion of the video stream of the software application or clinical application on the virtual information panel. The controller(s) can recognize text within a first predetermined region on a screen of the clinical application. In response to recognition of the text, controller(s) can trigger clipping of a second predetermined portion of the screen of the clinical application wherein the second predetermined portion comprises graphical or image information. The controller(s) can reproduce the graphical or image information from the second predetermined portion on the virtual information panel. The controller(s) can duplicate a navigation guidance region on the virtual information panel. The navigation guidance region can display one or more surgical objects tracked by a localizer of a surgical navigation system, or graphical representations thereof. The information can be provided by the software application or clinical application in a first format. In response to recognition of the information, the controller(s) can transform the information from the software application or clinical application into a second format adapted for the virtual information panel. The controller(s) can transform the information into the second format by automatically performing one or more of the following: re-arrange the information; crop the information; clip the information and/or re-size the information. The controller(s) can transmit, to a remote server, the information recognized from the video stream. The information can be transmitted for data analytic purposes. Virtual panels can be displayed as a series of sub-panels. For example, one virtual panel can be a full display of the clinical application, a first sub-panel can be a portion of the clinical application, and a second sub-panel can display a portion of the first-sub panel, etc. The sub-paneling can involve displaying each panel in front of the parent panel. Identified surgical context can trigger display of each sub-panel.

The connectivity system can include a housing. The housing can forma device separate from the host system/device or HMD. The computing system, input device and output device being supported by the housing. A mount can be attached to the housing to enable the housing to be mounted to a component of the host system/device, such as a display or a component of a movable cart of the host system/device. The input device can be a wired video signal input. The output device can include a wireless communicator to communicate with the HMD.

The HMD can include a camera configured to produce a live video stream of the real-world view. The controller(s) can combine the virtual object with the real-world view by combining the virtual object into the live video stream. The controller(s) can recognize the surgical information from the camera of the HMD. The HMD can include a transparent lens and can combine the virtual object with the real-world view by superimposing or overlaying the virtual object onto the transparent lens. The controller(s) can recognize surgical information from any camera source, such as: a navigation system camera, an endoscope, a laparoscope, an arthroscope, or a camera of the HMD.

The connectivity system can improve latency related to transmission of video data to the HMD by modelling the video data into a custom data type. The custom data type can utilize parameter sets for the video data. The parameter sets can include Sequence Parameter Sets (SPS), Video Parameter Sets (VPS), and Picture Parameter Sets (PPS). The video data can include a plurality of video frames and wherein the computing system is configured model each frame into the custom data type. The computing system can receive the video data as raw objects containing samples of the video data and a buffer of the video data. The computing system is further configured to encode the raw objects. The computing system is configured to model the encoded raw objects into the custom data type. The computing system can utilize a network protocol customized to facilitate wireless communication of the modeled video data to the HMD. The output device can be a WiFi router and the computing device can be coupled to the WiFi router using a wired connection, such as an ethernet cable.

### BRIEF DESCRIPTION OF THE DRAWINGS

Advantages of the present invention will be readily appreciated as the same becomes better understood by reference to the following detailed description when considered in connection with the accompanying drawings wherein:

FIG. 1 is a perspective view of a surgical system, according to one implementation.

FIG. 2 is a schematic view of an example control system can that be used with the surgical system.

FIG. 3 is an illustration of various coordinate systems and transforms that can be established relative to the various components of the surgical system, according to one implementation.

FIGS. 4A and 4B are diagrams illustrating interrelation between a world coordinate system, an HMD coordinate system, and a view coordinate system, according to one implementation.

FIGS. 5A, 5B and 5C are illustrations of example alignment guides that can be displayed by the HMD to assist a user to setting the pose of the view coordinate system.

FIG. 6 illustrates a sample first-person view through the HMD display wherein the user provides control inputs to select and manipulate the alignment guide for setting a pose of the view coordinate system, according to one implementation.

FIG. 7 illustrates a sample first-person view through the HMD display wherein the user provides control inputs to move a position guide object of the alignment guide relative to the target surgical site to set the position of the view coordinate system and to subsequently select an orientation guide object of the alignment guide, according to one implementation.

FIG. 8 illustrates a sample first-person view through the HMD display wherein the user provides control inputs to move the orientation guide object of the alignment guide to set the orientation of the view coordinate system, according to one implementation.

FIG. 9 illustrates a sample view of a bone preparation screen of a clinical application of the surgical navigation system, wherein certain information is identified on the screen using a stream analyzer, according to one implementation.

FIG. 10 illustrates a sample view of a bone registration screen of the clinical application of the surgical navigation system, wherein certain information is identified on the screen using the stream analyzer, according to one implementation.

FIG. 11 illustrates a sample first-person view through the HMD display wherein the HMD displays several virtual information panels in response to detection of surgical information related to bone registration, according to one implementation.

FIG. 12 illustrates a sample first-person view through the HMD display wherein the HMD displays a virtual information panel at a specific pose within the view coordinate system in response to detection of surgical information related to bone preparation, according to one implementation.

FIG. 13 illustrates a sample first-person view through the HMD display wherein the user provides control inputs to change a pose of the virtual information panel of FIG. 12, according to one implementation.

FIG. 14 is a flowchart of method steps that may be performed to configure and operate the extended reality system, according to one implementation.

FIG. 15 is a flowchart of method steps that may be performed to process video data from the software/clinical application for reducing latency in wireless transmission of the video data to the HMD.

### DETAILED DESCRIPTION

### I. Example System Overview

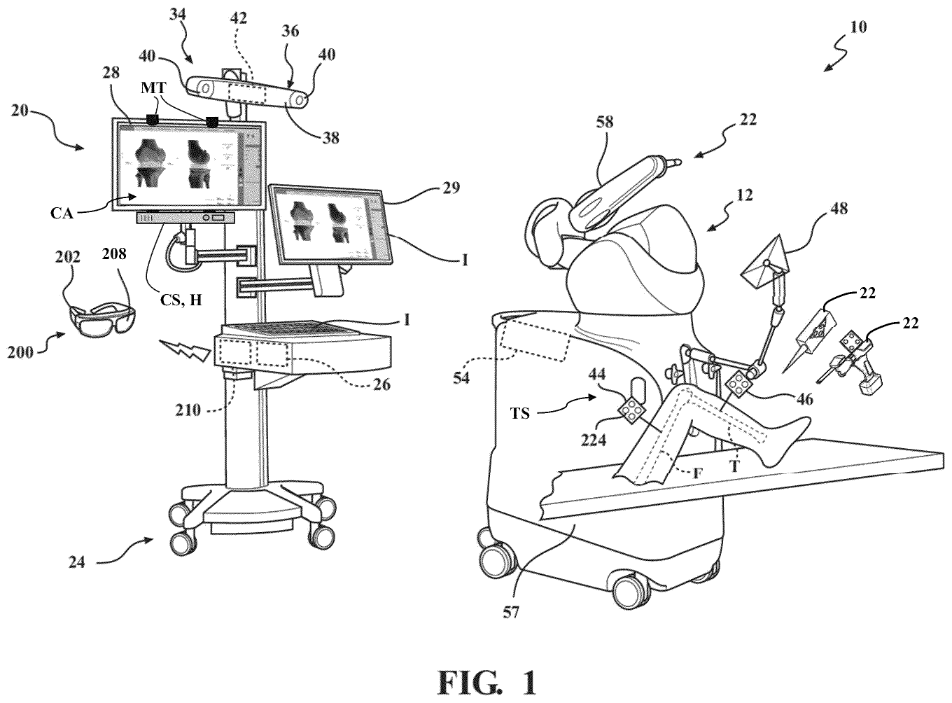

Referring to FIG. 1, a system 10 is provided. The system can be a surgical system 10 adapted for treating a patient. The surgical system 10 is shown in a surgical setting such as an operating room of a medical facility. The surgical system 10 may be used to perform any intraoperative surgical procedure on a patient. Example surgical procedures include, but are not limited to: partial knee arthroplasty, total knee arthroplasty, total hip arthroplasty, shoulder arthroplasty, spinal procedures, ankle procedures, endoscopic procedures, cranial procedures, lesion removal procedures, arthroscopic procedures, arthroscopic resection procedures, soft tissue or ligament repair procedures, neurological procedures, ENT procedures, minimally invasive MIS procedures, or the like. In the example shown in FIG. 1, the patient is undergoing a knee procedure. In addition, the following implementations describe the use of the surgical system 10 in performing a procedure in which material is removed from a femur F and/or a tibia T of a patient. However, it should be recognized that the surgical system 10 may be used to perform any suitable procedure in which material is removed from any suitable portion of a patient's anatomy, material is added to any suitable portion of the patient's anatomy (e.g., an implant, graft, etc.), and/or in which any other control of and/or visualization of a surgical tool is desired.

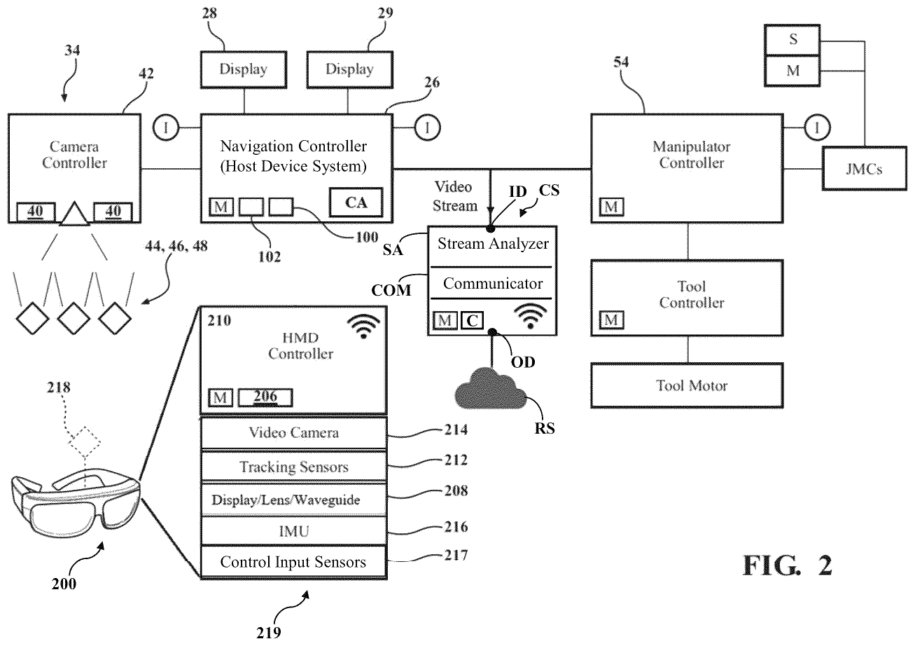

In the implementation shown, the surgical system 10 includes a manipulator 12 (e.g., surgical robot) and a navigation system 20. The navigation system 20 is set up to track movement of various objects in the operating room. Such objects include, for example, a surgical tool 22, a femur F of a patient, and a tibia T of the patient. The navigation system 20 tracks these objects for purposes of displaying their relative positions and orientations to the surgeon on a clinical application (CA) and, in some cases, for purposes of controlling or constraining movement of the surgical tool 22 relative to virtual cutting boundaries (VB) associated with the femur F and tibia T. An example control scheme for the surgical system 10 is shown in FIG. 2.

In the implementation shown, the surgical tool 22 is attached to the manipulator 12. Such an arrangement is shown in U.S. Pat. No. 9,119,655, entitled, “Surgical Manipulator Capable of Controlling a Surgical Instrument in Multiple Modes,” the disclosure of which is hereby incorporated by reference. In one example, the manipulator 12 has a base 57, a plurality of links 58 extending from the base 57, and a plurality of joints (not numbered) for moving the surgical tool 22 with respect to the base 57. The links 58 and joints form a robotic arm. Some or all of the joints may be passive joints or active joints. The manipulator 12 may have a serial arm or parallel arm configuration. The manipulator 12 can be floor mounted, ceiling mounted, gantry mounted, table mounted, or patient mounted. More than one manipulator 12 can be utilized.

While the surgical system 10 is illustrated in FIGS. 1-3 as including the surgical tool 22 attached to the manipulator 12, it should be recognized that the surgical system 10 may additionally or alternatively include one or more manually operated or hand-held surgical tools 22. For example, the surgical tool 22 may include a hand-held motorized saw, drill, bur, probe, or other suitable tool that may be held and manually operated by a surgeon. Any implementations described with reference to the use of the manipulator 12 may also apply to the use of a hand-held tool 22 with appropriate modifications.

The navigation system 20 includes one or more computer cart assemblies 24 that houses one or more navigation controllers 26. A navigation interface is in operative communication with the navigation controller 26. The navigation interface includes one or more displays 28, 29 adjustably mounted to the computer cart assembly 24 or mounted to separate carts as shown. Input devices I such as a keyboard and mouse can be used to input information into the navigation controller 26 or otherwise select/control certain aspects of the navigation controller 26. Other input devices I are contemplated including a touch screen, a microphone for voice-activation input, an optical sensor for gesture input, and the like.

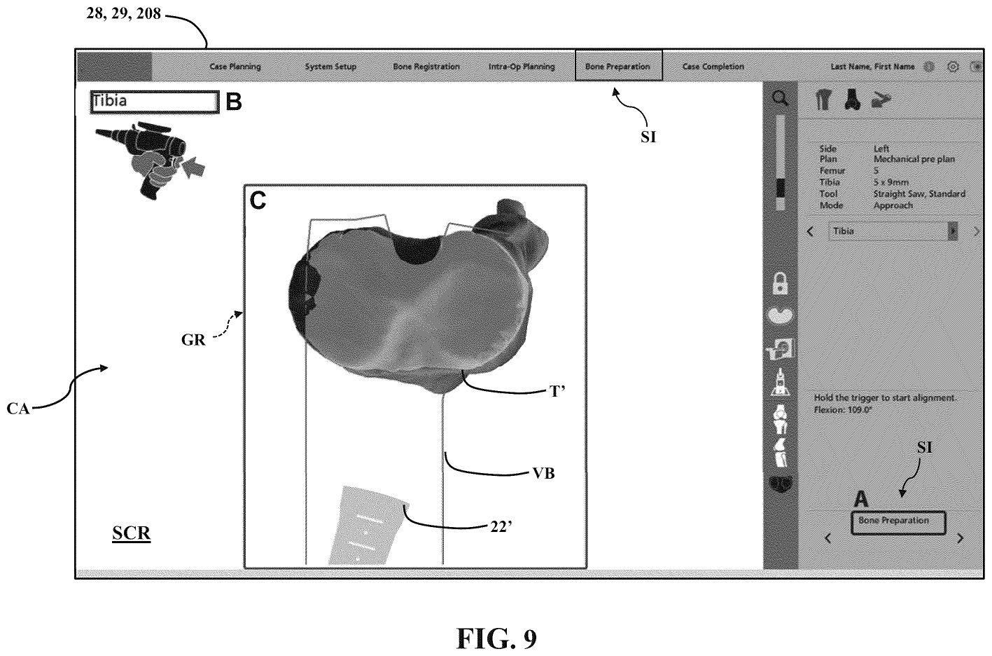

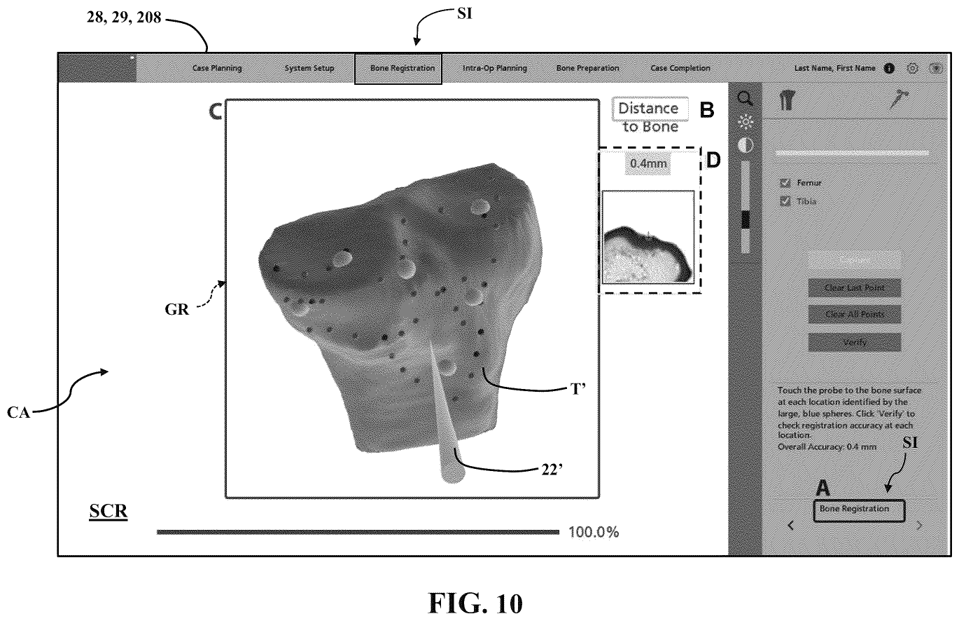

The clinical application CA can be displayed on one or more displays 28, 29 of the navigation system 20. The clinical application CA assists a surgeon or staff in performing the surgical procedure. The clinical application CA can have a plurality of different screens related to the surgical procedure. Such screens can include a pre-operative planning screen, an operating room setup screen, an anatomical registration screen, an intra-operative planning screen, an anatomical preparation screen, or a post-operative evaluation screen, and the like. The clinical application CA can present a navigation guidance region GR that displays one or more of the surgical objects tracked by a localizer 34 of the navigation system 20 (see FIGS. 9 and 10).

The localizer 34 communicates with the navigation controller 26. In the implementation shown, the localizer 34 is an optical localizer and includes a camera unit 36. The camera unit 36 has a housing 38 comprising an outer casing that houses one or more optical sensors 40. The optical sensors 40 can detect light signals, such as infrared (IR) signals and/or visible light signals. Camera unit 36 can be mounted on an adjustable arm to position the optical sensors 40 with a field-of-view of the below discussed trackers that, ideally, is free from obstructions. The camera unit 36 includes a camera controller 42 in communication with the optical sensors 40 to receive signals from the optical sensors 40. The camera controller 42 communicates with the navigation controller 26 through either a wired or wireless connection (not shown). In other implementations, the optical sensors 40 communicate directly with the navigation controller 26. Position and orientation signals and/or data are transmitted to the navigation controller 26 for purposes of tracking objects. The computer cart assembly 24, display 28, and camera unit 36 may be like those described in U.S. Pat. No. 7,725,162 to Malackowski, et al. issued on May 25, 2010, entitled “Surgery System,” the disclosure of which is hereby incorporated by reference. The navigation controller 26 can be a personal computer or laptop computer. Navigation controller 26 includes the displays 28, 29, central processing unit (CPU) and/or other processors, memory (not shown), and storage (not shown). The navigation controller 26 is loaded with software that converts the signals received from the camera unit 36 into data representative of the position and orientation of the objects being tracked. The navigation controller 26 includes a navigation processor. It should be understood that the navigation processor could include one or more processors to control operation of the navigation controller 26. The processors can be any type of microprocessor or multi-processor system. The term processor is not intended to limit the scope of any implementation to a single processor.

Navigation system 20 is operable with a plurality of tracking devices 44, 46, 48, also referred to herein as trackers. In the illustrated implementation, one tracker 44 can be firmly affixed to the femur F of the patient and another tracker 46 can be firmly affixed to the tibia T of the patient. Trackers 44, 46 are firmly affixed to sections of bone in an implementation. For example, trackers 44, 46 may be attached to the femur F and tibia T in the manner shown in U.S. Pat. No. 7,725,162 to Malackowski, et al. issued on May 25, 2010, entitled “Surgery System,” the disclosure of which is hereby incorporated by reference. Trackers 44, 46 may also be mounted like those shown in U.S. patent application Ser. No. 14/156,856, filed on Jan. 16, 2014, entitled, “Navigation Systems and Methods for Indicating and Reducing Line-of-Sight Errors,” hereby incorporated by reference herein. The trackers 44, 46 may be mounted to other tissue types or parts of the anatomy. A tool tracker 48 can be coupled to the manipulator 12 or the tool 22 at any suitable location. The tool tracker 48 can be integrated into the surgical tool 22 during manufacture or may be separately mounted to the surgical tool 22 (or to an end effector attached to the manipulator 12 of which the surgical tool 22 forms a part) in preparation for surgical procedures. The working end of the surgical tool 22, which is being tracked by virtue of the tool tracker 48, may be referred to herein as an energy applicator, and may be a rotating bur, saw, router, reamer, impactor, electrical ablation device, cut guide, tool holder, probe, or the like.

In one implementation, optical sensors 40 of the localizer 34 receive light signals from the trackers 44, 46, 48. In one example, the trackers 44, 46, 48 are passive trackers. In this implementation, each tracker 44, 46, 48 has at least three passive tracking elements or markers (e.g., reflectors) for transmitting light signals (e.g., reflecting light emitted from the camera unit 36) to the optical sensors 40. In other implementations, active tracking markers can be employed. The active markers can be, for example, light emitting diodes transmitting light, such as infrared light. Active and passive arrangements are possible. The camera unit 36 receives optical signals from the trackers 44, 46, 48 and outputs to the navigation controller 26 signals relating to the position of the tracking markers of the trackers 44, 46, 48 relative to the localizer 34. Based on the received optical signals, navigation controller 26 generates data indicating the relative positions and orientations of the trackers 44, 46, 48 relative to the localizer 34. These relative positions can be displayed on the clinical application CA as graphical representations for surgical guidance.

In another implementation, the navigation system 20 and/or the localizer 34 are radio frequency (RF) based. For example, the navigation system 20 may comprise an RF transceiver coupled to the navigation controller 26. Here, the trackers 44, 46, 48 may comprise RF emitters or transponders, which may be passive or may be actively energized. The RF transceiver transmits an RF tracking signal, and the RF emitters respond with RF signals such that tracked states are communicated to (or interpreted by) the navigation controller 26. The RF signals may be of any suitable frequency. The RF transceiver may be positioned at any suitable location to track the objects using RF signals effectively. Furthermore, examples of RF-based navigation systems may have structural configurations that are different than the navigation system 20 illustrated throughout the drawings.

In other examples, the navigation system 20 and/or localizer 34 are electromagnetically (EM) based. For example, the navigation system 20 may comprise an EM transceiver coupled to the navigation controller 26. Here, the trackers 44, 46, 48 may comprise EM components attached thereto (e.g., various types of magnetic trackers, electromagnetic trackers, inductive trackers, and the like), which may be passive or may be actively energized. The EM transceiver generates an EM field, and the EM components respond with EM signals such that tracked states are communicated to (or interpreted by) the navigation controller 26. The navigation controller 26 may analyze the received EM signals to associate relative states thereto. Here too, examples of EM-based navigation systems may have structural configurations that are different than the navigation system 20 illustrated throughout the drawings.

In other examples, the navigation system 20 and/or the localizer 34 could be based on one or more other types of tracking systems. For example, an ultrasound-based tracking system coupled to the navigation controller 26 could be provided to facilitate acquiring ultrasound images of markers that define trackable features on the tracked objects such that tracked states are communicated to (or interpreted by) the navigation controller 26 based on the ultrasound images. By way of further example, a fluoroscopy-based imaging system (e.g., a C-arm) coupled to the navigation controller 26 could be provided to facilitate acquiring X-ray images of radio-opaque markers that define trackable features such that tracked states are communicated to (or interpreted by) the navigation controller 26 based on the X-ray images.

Furthermore, in some examples, a machine-vision tracking system, including a vision camera can be coupled to the navigation controller 26 and could be provided to facilitate acquiring 2D and/or 3D machine-vision images of structural features that define trackable features such that tracked states TS are communicated to (or interpreted by) the navigation controller 26 based on the machine-vision images. The machine vision system can be integrated into the camera unit 36, optionally in combination with infrared sensors. The machine vision system can create depth maps and can detect objects with or without trackers. The machine vision system can detect patterns, shapes, colors, computer-codes, tracking geometries, or the like.

Various types of tracking and/or imaging systems could define the localizer 34 and/or form a part of the navigation system 20 without departing from the scope of the present disclosure. Furthermore, the navigation system 20 and/or localizer 34 may have other suitable components or structure not specifically recited herein, and the various techniques, methods, and/or components described herein with respect to the optically-based navigation system 20 shown throughout the drawings may be implemented or provided for any of the other examples of the navigation system 20 described herein. For example, the navigation system 20 may utilize solely inertial tracking and/or combinations of different tracking techniques, sensors, and the like. Other configurations are contemplated.

Based on the position and orientation of the trackers 44, 46, 48 and previously loaded data, navigation controller 26 can determine the position of the working end of the surgical tool 22 (e.g., the centroid of a surgical bur) and/or the orientation of the surgical tool 22 relative to the tissue against which the working end is to be applied. In some implementations, the navigation controller 26 forwards these data to a manipulator controller 54. The manipulator controller 54 can then use the data to control the manipulator 12. This control can be like that described in U.S. Pat. No. 9,119,655, entitled, “Surgical Manipulator Capable of Controlling a Surgical Instrument in Multiple Modes,” or like that described in U.S. Pat. No. 8,010,180, entitled, “Haptic Guidance System and Method”, the disclosures of which are hereby incorporated by reference.

In one implementation, the manipulator 12 is controlled to stay within a preoperatively defined virtual boundary VB that can be determined by a surgical plan. The virtual boundary VB may be a virtual cutting boundary which defines the material of the anatomy (e.g., the femur F and tibia T) to be removed by the surgical tool 22. More specifically, each of the femur F and tibia T has a target volume of material that is to be removed by the working end of the surgical tool 22. The target volumes are defined by one or more virtual cutting boundaries. The virtual cutting boundaries define the surfaces of the bone that should remain after the procedure. The navigation system 20 tracks and controls the surgical tool 22 to ensure that the working end, e.g., the surgical bur, removes the target volume of material and does not extend beyond the virtual cutting boundary, as disclosed in U.S. Pat. No. 9,119,655, entitled, “Surgical Manipulator Capable of Controlling a Surgical Instrument in Multiple Modes,” the disclosure of which is hereby incorporated by reference, or as disclosed in U.S. Pat. No. 8,010,180, entitled, “Haptic Guidance System and Method”, the disclosure of which is hereby incorporated by reference.

The virtual cutting boundary VB may be defined within a virtual model of the anatomy (e.g., the femur F and tibia T), or separately from the virtual model. The virtual cutting boundary may be represented as a mesh surface, constructive solid geometry (CSG), voxels, or using other boundary representation techniques. The surgical tool 22 may be used to cut away material from the femur F and tibia T to receive an implant. The surgical implants may include unicompartmental, bicompartmental, or total knee implants as shown in U.S. Pat. No. 9,381,085, entitled, “Prosthetic Implant and Method of Implantation,” the disclosure of which is hereby incorporated by reference. Other implants, such as hip implants, shoulder implants, spine implants, and the like are also contemplated. The focus of the description on knee implants is provided as one example. These concepts can be equally applied to other types of surgical procedures, including those performed without placing implants.

The navigation controller 26 also generates image signals that indicate the relative position of the working end to the tissue. These image signals are applied to the displays 28, 29. The displays 28, 29, based on these signals, generate images on the clinical application CA that allow the surgeon and staff to view the relative position of the working end to the target site TS.

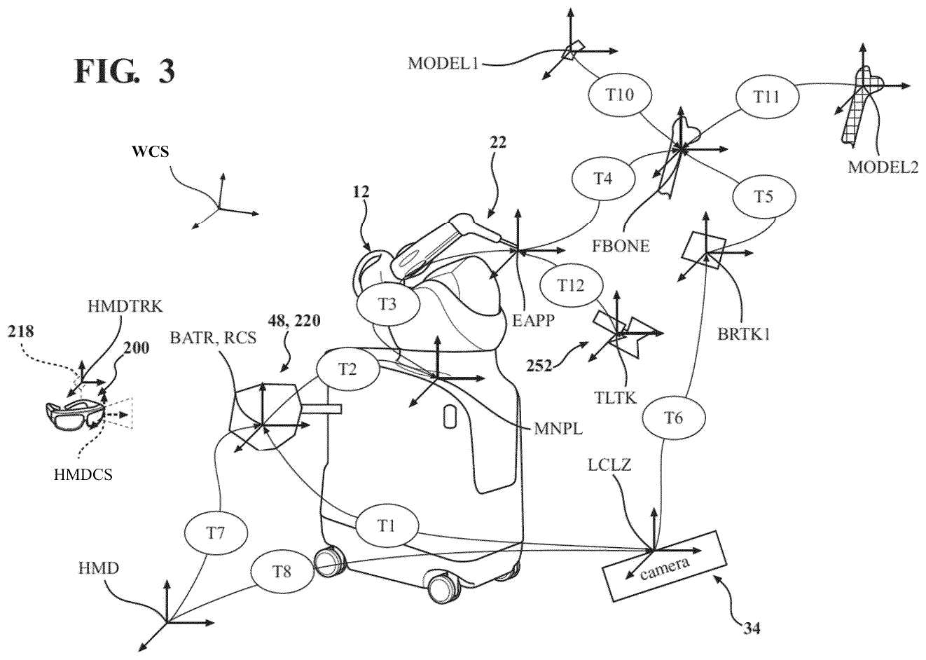

Referring to FIG. 3, tracking of objects can be conducted with reference to a localizer coordinate system LCLZ. The localizer coordinate system has an origin and an orientation (a set of x, y, and z planes). Each tracker 44, 46, 48 and object being tracked also has its own coordinate system separate from the localizer coordinate system LCLZ. Components of the navigation system 20 that have their own coordinate systems are the bone trackers 44, 46 (one of which is shown in FIG. 3) and the base tracker 48. These coordinate systems are represented as, respectively, bone tracker coordinate systems BTRK1, BTRK2 (BTRK1 shown), and base tracker coordinate system BATR. The world coordinate system WCS indicates the coordinate system of the real-world, or room, in which the objects are located.

Navigation system 20 monitors the positions of the femur F and tibia T of the patient by monitoring the position of bone trackers 44, 46 rigidly attached to bone. Femur coordinate system is FBONE and tibia coordinate system is TBONE, which are the coordinate systems of the bones to which the bone trackers 44, 46 are rigidly attached.

Prior to the start of the intraoperative procedure, preoperative images of the femur F and tibia T may be generated (or of other portions of the anatomy in other implementations). The preoperative images can be stored as two-dimensional or three-dimensional patient image data in a computer-readable storage device, such as memory within the navigation system 20. The patient image data may be based on X-ray scans or computed tomography (CT) scans of the patient's anatomy. The patient image data may then be used to generate two-dimensional images or three-dimensional models of the patient's anatomy. The pre-operative data and models may be used for purposes of surgical planning purposes and intraoperative guidance. For example, the surgical plan (e.g., tool path TP or resection volume or boundaries VB), may be planned relative to the virtual model. The virtual model and surgical plan can then be registered to the anatomy using any appropriate registration technique, such as pointer registration, imageless registration, or the like.

In preparation for the intraoperative procedure, the images or three-dimensional models developed from the image data are mapped to the femur coordinate system FBONE and tibia coordinate system TBONE (see transform T11). One of these models is shown in FIG. 3 with model coordinate system MODEL2. These images/models are fixed in the femur coordinate system FBONE and tibia coordinate system TBONE. As an alternative to taking preoperative images, plans for treatment can be developed in the operating room (OR) from kinematic studies, bone tracing, and other methods. The models described herein may be represented by mesh surfaces, constructive solid geometry (CSG), voxels, or using other model constructs.

During an initial phase of the intraoperative procedure, the bone trackers 44, 46 are coupled to the bones of the patient. The pose (position and orientation) of coordinate systems FBONE and TBONE are mapped to coordinate systems BTRK1 and BTRK2, respectively (see transform T5). In one implementation, a pointer instrument 252 (TLTK), such as disclosed in U.S. Pat. No. 7,725,162 to Malackowski, et al., hereby incorporated by reference, having its own tracker, may be used to register the femur coordinate system FBONE and tibia coordinate system TBONE to the bone tracker coordinate systems BTRK1 and BTRK2, respectively. Given the fixed relationship between the bones and their bone trackers 44, 46, positions and orientations of the femur F and tibia T in the femur coordinate system FBONE and tibia coordinate system TBONE can be transformed to the bone tracker coordinate systems BTRK1 and BTRK2 so the localizer 34 is able to track the femur F and tibia T by tracking the bone trackers 44, 46. These pose-describing data can be stored in memory integral with both manipulator controller 54 and navigation controller 26.

The working end of the surgical tool 22 has its own coordinate system. In some implementations, the surgical tool 22 comprises a handpiece and an accessory that is removably coupled to the handpiece. The accessory may be referred to as the energy applicator and may comprise a bur, an electrosurgical tip, an ultrasonic tip, or the like. Thus, the working end of the surgical tool 22 may comprise the energy applicator. The coordinate system of the surgical tool 22 is referenced herein as coordinate system EAPP. The origin of the coordinate system EAPP may represent a centroid of a surgical cutting bur, for example. In other implementations, the accessory may simply comprise a probe or other surgical tool with the origin of the coordinate system EAPP being a tip of the probe. The pose of coordinate system EAPP is registered to the pose of base tracker coordinate system BATR before the procedure begins (see transforms T1, T2, T3). Accordingly, the poses of these coordinate systems EAPP, BATR relative to each other are determined. The pose-describing data can be stored in memory integral with both manipulator controller 54 and navigation controller 26.

Referring to FIG. 2, a localization engine 100 is a software module that can be considered part of the navigation system 20. Components of the localization engine 100 run on navigation controller 26. In some implementations, the localization engine 100 may run on the manipulator controller 54. Localization engine 100 receives as inputs the signals from the localizer 34 and, in some implementations, signals from the tracker controller. Based on these signals, localization engine 100 can determine the pose of the bone tracker coordinate systems BTRK1 and BTRK2 in the localizer coordinate system LCLZ (see transform T6). Based on the same signals received for the base tracker 48, the localization engine 100 determines the pose of the base tracker coordinate system BATR in the localizer coordinate system LCLZ (see transform T1).

The localization engine 100 forwards the signals representative of the poses of trackers 44, 46, 48 to a coordinate transformer 102. Coordinate transformer 102 is a navigation system software module that runs on navigation controller 26. Coordinate transformer 102 references the data that defines the relationship between the preoperative images of the patient and the bone trackers 44, 46. Coordinate transformer 102 can also store the data indicating the pose of the working end of the surgical tool 22 relative to the base tracker 48.

During the procedure, the coordinate transformer 102 receives the data indicating the relative poses of the trackers 44, 46, 48 to the localizer 34. Based on these data, the previously loaded data, and the below-described encoder data from the manipulator 12, the coordinate transformer 102 can generate data indicating the relative positions and orientations of the coordinate system EAPP and the bone coordinate systems, FBONE and TBONE. As a result, coordinate transformer 102 generates data indicating the position and orientation of the working end of the surgical tool 22 relative to the tissue (e.g., bone) against which the working end is applied. Image signals representative of these data are forwarded to displays 28, 29 enabling the surgeon and staff to view this information. In certain implementations, other signals representative of these data can be forwarded to the manipulator controller 54 to guide the manipulator 12 and corresponding movement of the surgical tool 22.

The manipulator 12 has the ability to operate in a manual mode or a semi-autonomous mode in which the surgical tool 22 is moved along a predefined tool path, as described in U.S. Pat. No. 9,119,655, entitled, “Surgical Manipulator Capable of Controlling a Surgical Instrument in Multiple Modes,” the disclosure of which is hereby incorporated by reference, or the manipulator 12 may be configured to move in the manner described in U.S. Pat. No. 8,010,180, entitled, “Haptic Guidance System and Method”, the disclosure of which is hereby incorporated by reference.

The manipulator controller 54 can use the position and orientation data of the surgical tool 22 and the patient's anatomy to control the manipulator 12 as described in U.S. Pat. No. 9,119,655, entitled, “Surgical Manipulator Capable of Controlling a Surgical Instrument in Multiple Modes,” the disclosure of which is hereby incorporated by reference, or to control the manipulator 12 as described in U.S. Pat. No. 8,010,180, entitled, “Haptic Guidance System and Method”, the disclosure of which is hereby incorporated by reference.

The manipulator controller 54 may have a central processing unit (CPU) and/or other manipulator processors, memory (not shown), and storage (not shown). The manipulator controller 54, also referred to as a manipulator computer, is loaded with software as described below. The manipulator processors could include one or more processors to control operation of the manipulator 12. The processors can be any type of microprocessor or multi-processor system. The term processor is not intended to limit any implementation to a single processor.

A plurality of position sensors S are associated with the plurality of links 58 of the manipulator 12. In one implementation, the position sensors S are encoders. The position sensors S may be any suitable type of encoder, such as rotary encoders. Each position sensor S is associated with a joint actuator, such as a joint motor M. Each position sensor S is a sensor that monitors the angular position of one of six motor driven links 58 of the manipulator 12 with which the position sensor S is associated. Multiple position sensors S may be associated with each joint of the manipulator 12 in some implementations. The manipulator 12 can also include a force/torque sensor coupled between the distal end of the manipulator 12 and the end effector for detecting manual forces/torques exerted on the tool 22 by an operator. The input forces/torques can be used to command movement of the manipulator 12 and/or to detect collisions with the tool 22.

In some modes, the manipulator controller 54 determines the desired location to which the surgical tool 22 should be moved. Based on this determination, and information relating to the current location (e.g., pose) of the surgical tool 22, the manipulator controller 54 determines the extent to which each of the plurality of links 58 needs to be moved in order to reposition the surgical tool 22 from the current location to the desired location. The data regarding where the plurality of links 58 are to be positioned is forwarded to joint motor controllers JMCs that control the joints of the manipulator 12 to move the plurality of links 58 and thereby move the surgical tool 22 from the current location to the desired location. In other modes, the manipulator 12 is capable of being manipulated as described in U.S. Pat. No. 8,010,180, entitled, “Haptic Guidance System and Method”, the disclosure of which is hereby incorporated by reference, in which case the actuators are controlled by the manipulator controller 54 to provide gravity compensation to prevent the surgical tool 22 from lowering due to gravity and/or to activate in response to a user attempting to place the working end of the surgical tool 22 beyond a virtual boundary VB.

In order to determine the current location of the surgical tool 22, data from the position sensors S is used to determine measured joint angles. The measured joint angles of the joints are forwarded to a forward kinematics module, as known in the art. Based on the measured joint angles and preloaded data, the forward kinematics module determines the pose of the surgical tool 22 in a manipulator coordinate system MNPL (see transform T3 in FIG. 3). The preloaded data are data that define the geometry of the plurality of links 58 and joints. With this encoder-based data, the manipulator controller 54 and/or navigation controller 26 can transform coordinates from the localizer coordinate system LCLZ into the manipulator coordinate system MNPL, vice versa, or can transform coordinates from one coordinate system into any other coordinate system described herein using transformation techniques. In many cases, the coordinates of interest associated with the surgical tool 22 (e.g., the tool center point or TCP), the virtual boundaries, and the tissue being treated, are transformed into a common coordinate system for purposes of relative tracking and display.

In the implementation shown in FIG. 3, transforms T1-T6 are utilized to transform relevant coordinates into the femur coordinate system FBONE so that the position and/or orientation of the surgical tool 22 can be tracked relative to the position and orientation of the femur (e.g., the femur model) and/or the position and orientation of the volume of material to be treated by the surgical tool 22 (e.g., a cut-volume model: see transform T10). The relative positions and/or orientations of these objects can also be represented on the displays 28, 29 to enhance the user's visualization before, during, and/or after surgery.

While the example surgical system 10 has been described with reference to the Figures, the surgical system 10 is not intended to be limited to what is specifically shown and described. For example, the surgical system 10 may not include the manipulator 12 or the navigation system 20 as specifically shown. Other systems are contemplated without departing from the scope of the disclosure.

### II. Head Mounted Device

Referring back to FIGS. 1 and 2, one or more head-mounted devices (HMDs) 200 may be incorporated into the surgical system 10. The HMD may be employed to enhance visualization before, during, and/or after surgery. The HMD 200 is an extended reality device, which can include aspects of augmented reality, mixed reality, virtual reality, and the like. The HMD 200 can be used to visualize the same objects previously described as being visualized on the displays 28, 29, and can also be used to visualize other objects, features, instructions, warnings, etc. The HMD 200 can be used to assist with visualization of the volume of material to be cut from the patient, to help visualize the size of implants and/or to place implants for the patient, to assist with registration and calibration of objects being tracked via the navigation system 20, to see instructions and/or warnings, among other uses, as described further below.

The HMD 200 has a display 208 onto which computer-generated content can be displayed onto a real-world view. In the implementation described herein, the HMD 200 provides on the HMD display 208 a computational holographic/superimposed/overlay of computer-generated content over the real-world view. In one example, the real-world view is acquired by a video camera 214 attached to the HMD. The video camera 214 produces a live video stream of the real-world and the computer-generated content may be combined into video stream of the real world. In such instances, the HMD display 208 may include one or more high-resolution displays positioned in front of the user's eyes. The HMD display 208 may be opaque in such scenarios.

In other implementations, the HMD 200 may implement natural see-through techniques whereby the HMD display 208 is implemented as a transparent lens/visor/waveguide provided between the user's eyes and the real-world. The real-world view is acquired naturally by the user's eyes and the computer-generated content is provided on the transparent lens/visor/waveguide. Such see-through techniques can include a diffractive waveguide, holographic waveguide, polarized waveguide, reflective waveguide, or switchable waveguide.

The HMD 200 includes a support structure 202, which may be head-mountable in the form of an eyeglass or glasses, headwear or headset, or eyewear (such as a digital contact lens or lenses). The HMD 200 may include additional headbands or supports to hold the HMD 200 on the user's head. In other implementations, the HMD 200 may be integrated into a surgical helmet or other structure worn on the user's head, neck, and/or shoulders. Although not shown, it is contemplated that instead of the HMD 200, an extended reality display screen, such as a monitor, tablet, or hand-held display may be used, which can include similar hardware and capabilities as the described HMD 200.

The HMD 200 can include an HMD controller 210. The HMD controller 210 can include a content generator 206 that generates the computer-generated content (also referred to as virtual images) and that transmits those images to the user through the HMD display 208. The HMD controller 210 controls the transmission of the computer-generated content to the HMD display 208. The HMD controller 210 may be a separate computer, located remotely from the support structure 202 of the HMD 200, or may be integrated into the support structure 202 of the HMD 200. The HMD controller 210 may be a laptop computer, desktop computer, microcontroller, or the like with memory, one or more processors (e.g., multi-core processors), input devices I, output devices (fixed display in addition to HMD 200), storage capability, etc.

The HMD 200 comprises a plurality of tracking sensors 212 that are in communication with the HMD controller 210. In some cases, the tracking sensors 212 are provided to establish a global coordinate system for the HMD 200, also referred to as an HMD coordinate system. The HMD coordinate system is established by these tracking sensors 212, which may comprise camera sensors or other sensor types, in some cases combined with IR depth sensors, to layout the space surrounding the HMD 200, such as using structure-from-motion techniques or the like. The HMD 200 can also comprise a photo/video camera 214 in communication with the HMD controller 210. The camera 214 may be used to obtain photographic images or video with the HMD 200, which can be useful in identifying objects or markers attached to objects, as will be described further below. The HMD 200 can comprise an inertial measurement unit IMU 216 in communication with the HMD controller 210. The IMU 216 may comprise one or more 3-D accelerometers, 3-D gyroscopes, and the like to assist with determining a position and/or orientation of the HMD 200 in the HMD coordinate system or to assist with tracking relative to other coordinate systems. The HMD 200 could have a speaker to generate a sound or vibrate to provide an indication to the HMD user of a warning or other information of relevance.

The HMD 200 may also comprise one or more control input sensors 217. In one example, the control input sensors 217 are configured to recognize gesture or eye-based commands from the user. When detecting hand-gestures, the control input sensor 217 is able to sense the user's hands, fingers, or other objects for purposes of determining the user's gesture command and controlling the HMD 200, HMD controller 210, navigation controller 26, and/or manipulator controller 54 accordingly. Gesture commands can be used for any type of input used by the system 10. The gesture commands may be detected below the HMD 200 or may be detected by the camera 214 in front of the HMD 200. The control input sensor 217 to detect gesture can include one or more cameras, infrared sensors, motion sensors, or the like. Gesture controls can include any type of hand or finger motion, including but not limited to: pinching, pointing, swiping, circling, grasping, twisting, or the like. When detecting eye-based commands, the control input sensor 217 is able to sense the user's eye position, motion, dwell time (stare), gaze and the like, for purposes of determining the user's intended command and controlling the HMD 200, HMD controller 210, navigation controller 26, and/or manipulator controller 54 accordingly. The eye-based commands may be detected using an eye-tracker that is positioned to face the user's eyes, e.g., in front of the HMD display 208. Eye-based controls can include any type of eye-command, including but not limited to: selecting an object, moving an object, or the like. In one example, the user can select a computer-generated object displayed by the HMD 200 by staring at the object continuously for a threshold amount of time. The HMD can also control input sensors 217 in the form of a microphone for recording verbal commands. The HMD controller 210 can process the verbal commands and control the HMD display 208 in response.

Any of the described components of the HMD 200 that can sense information or process sensed information (including but not limited to, the HMD controller 210, the video camera 214, tracking sensors 212, IMU 216, and/or control input sensors 217) can be understood as being part of a “sensing system” of the HMD 220. The sensing system is identified by numeral 219 in FIG. 2.

The HMD 200 can be registered to one or more objects used in the operating room, such as the tissue being treated, the surgical tool 22, the manipulator 12, the trackers 44, 46, 48, the localizer 34, and/or the like. In one implementation, a local coordinate system HMDCS is associated with the HMD 200 to move with the HMD 200 so that the HMD 200 is fixed in a known position and orientation in the HMD coordinate system. The HMD 200 can utilize the tracking sensors 212 to map the surroundings and establish the HMD coordinate system. The HMD 200 can then utilize the camera 214 to find objects in the HMD coordinate system. In some implementations, the HMD 200 uses the camera 214 to capture video images of markers attached to the objects and then determines the location of the markers in the local coordinate system HMDCS of the HMD 200 using motion tracking techniques and then converts (transforms) those coordinates to the HMD coordinate system.

In another implementation, a separate HMD tracker 218 (see FIGS. 2 and 3), similar to the trackers 44, 46, 48, could be mounted to the HMD 200 (e.g., fixed to the support structure 202). The HMD tracker 218 can have its own HMD tracker coordinate system HMDTRK that is in a known position/orientation relative to the local coordinate system HMDCS of the HMD 200. Alternatively, the tracker coordinate system HMDTRK could be calibrated to the local coordinate system HMDCS using calibration techniques. In this implementation, the local coordinate system HMDCS becomes the HMD coordinate system and the transforms T7 and T8 would instead originate therefrom. The localizer 34 could then be used to track movement of the HMD 200 via the HMD tracker 218 and transformations could then easily be calculated to transform coordinates in the local coordinate system HMDCS to the localizer coordinate system LCLZ, the femur coordinate system FBONE, the manipulator coordinate system MNPL, or other coordinate system.

Referring back to FIG. 3, a registration device 220 may be provided with a plurality of registration markers 224 (shown in FIG. 1) to facilitate registering the HMD 200 to the localizer coordinate system LCLZ. The HMD 200 locates the registration markers 224 on the registration device 220 in the HMD coordinate system via the camera 214 thereby allowing the HMD controller 210 to create a transform T7 from the registration coordinate system RCS to the HMD coordinate system. The HMD controller 210 then needs to determine where the localizer coordinate system LCLZ is with respect to the HMD coordinate system so that the HMD controller 210 can generate images having a relationship to objects in the localizer coordinate system LCLZ or other coordinate system. The registration device 220 or any technique for registering and/or calibrating the HMD 200 to another coordinate system can be like that described in U.S. Pat. No. 10,499,997, entitled “Systems and Methods for Surgical Navigation”, the entire contents of which are hereby incorporated by reference in their entirety.

During use, for example, the localizer 34 and/or the navigation controller 26 can send data on an object (e.g., the cut volume model) to the HMD 200 so that the HMD 200 knows where the object is in the HMD coordinate system and can display an appropriate content in the HMD coordinate system. Any of the transforms T1-T12 can be combined to define or register the HMD coordinate system to any object. Once registration is complete, then the HMD 200 can be used to visualize computer-generated content in desired locations with respect to any objects in the operating room. Although these transforms have been described in detail, it is understood that the HMD 200 can operate without requiring any such transforms. The HMD 200 can display content without registering to the bone, or any part of the surgical system 10.

### III. Techniques Involving the HMD

Having introduced the system 10 and HMD 200 above, this section now describes various systems, methods, software, and techniques involving the HMD 200 including: (1) presentation and use of an alignment guide (AG) on the HMD 200 to assist a user in setting a pose of a view coordinate system VCS or a virtual object VO within the view coordinate system VCS; (2) a “smart” view technique whereby relevant information is automatically provided on the HMD 200 at appropriate times and locations, (3) a compatibility or connectivity technique or module that enables the HMD 200 to be seamlessly integrated with any host system/device that provides information on a software application and (4) techniques for reducing latency in communication of video data to the HMD 200. The above list of features introduces a selection of concepts in a simplified form that are further described below. The above list is not intended to limit the scope of the claimed subject matter nor identify key features or essential features of the claimed subject matter.

### A. Alignment Guide and Custom View Coordinate System

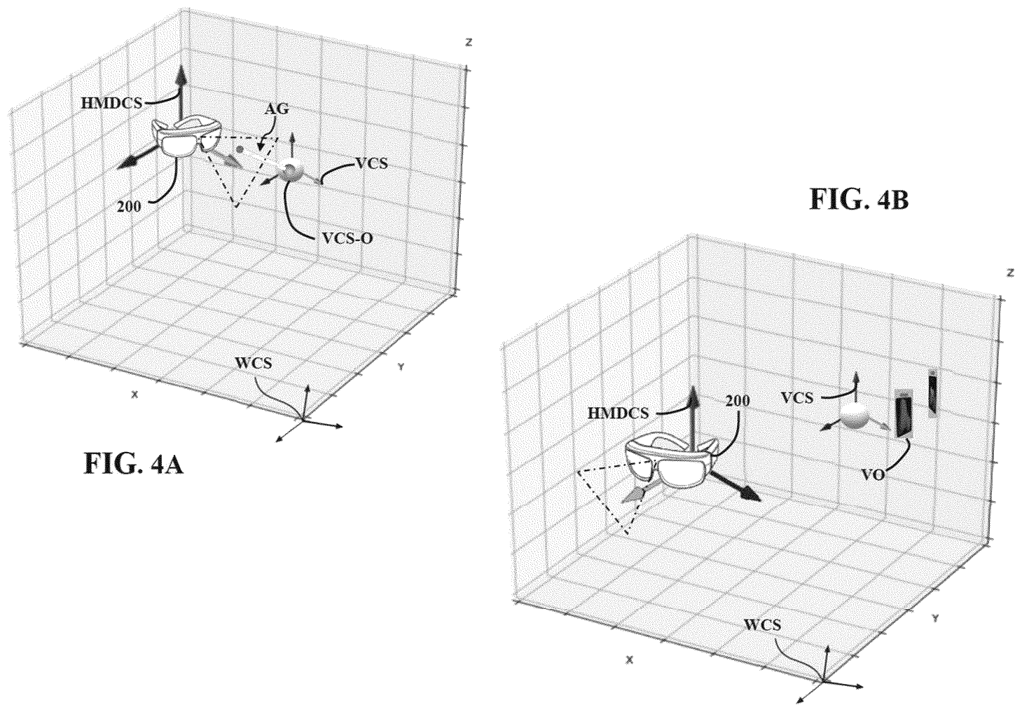

With reference to FIGS. 4-8, an alignment guide AG will be described in detail. The alignment guide AG is presented on the HMD display 208 to assist a user in setting a pose of a view coordinate system VCS. The view coordinate system VCS is a coordinate system that is calibrated or established by a user of the HMD 200 to enable presentation of a virtual object(s) VO (e.g., xR graphics) within this specifically calibrated view. The virtual objects VO will be described in detail below.

As will be understood from the described below, the alignment guide AG provides technical solutions not addressed by conventional xR systems. The alignment guide AG provides the user with intuitive and easily controllable means for customizing the VCS pose for placement of virtual object(s) VO provided by the HMD display 208. The alignment guide AG can be particularly useful for novice users who lack experience with complex extended reality controls. The alignment guide AG provides a precise view scheme for the user thereby avoiding a “one-size-fits-all” scheme as provided by conventional xR systems. The alignment guide AG is well-adapted for surgical purposes, whereby the VCS and virtual object(s) VO presented therein can be customized to the surgeon's preference relative to the target site TS to avoid disruption or distraction to the surgeon or the surgical procedure. The alignment guide AG can help set the pose of the VCS at a height, position and orientation that are ergonomically optimized to the specific height and viewing posture of the user/surgeon or to the surgical procedure.

FIGS. 4A and 4B are diagrams illustrating interrelation between the world coordinate system WCS, the HMD coordinate system HMDTRK, and the view coordinate system VCS, according to one implementation. The world coordinate system WCS indicates the coordinate system of the real-world, or room, in which the objects are located. The world coordinate system WCS is fixed. The HMD coordinate system HMDCS indicates the coordinate system that is fixed to the HMD 200 and changes pose based on relative pose changes of the HMD 200. The view coordinate system VCS is coordinate system that is separate from WCS and HMDCS. The view coordinate system VCS has a pose (i.e., position and/or orientation) that is set by the user of the HMD 200. As shown in FIG. 4A, the alignment guide AG can be specifically used for setting the pose of the view coordinate system VCS. Prior to, or during, calibration of the VCS, the alignment guide AG can be presented in the HMDCS such that the alignment guide AG moves with corresponding movement of the HMD 200 and remains in the user's view. This is to enable the user to continually see the alignment guide AG to perform the calibration process. Once the pose of the view coordinate system VCS is set using the alignment guide AG, the VCS pose can be defined and/or fixed relative to the world coordinate system WCS (as shown in FIG. 4A). As shown in FIG. 4B, after the pose of the view coordinate system VCS is established, the VCS can become defined and/or fixed to the world coordinate system WCS despite any relative change in pose of the HMD 200 and its coordinate system HMDCS. Therefore, if the user of the HMD 200 were to look entirely away from the view coordinate system VCS (as shown in FIG. 4B), the virtual objects VO within the view coordinate system VCS will not be visible to the user of the HMD 200. If the user of the HMD 200 were to look back at the view coordinate system VCS (such as shown in FIG. 4A), the virtual objects VO within the view coordinate system VCS will become visible to the user of the HMD 200. Hence, the view coordinate system VCS is clearly distinguishable from the mere view of the HMD 200. Of course, there may be other objects/views/displays that could be fixed and displayed in the HMDCS coordinate system so they remain visible on the HMD display 208 to the HMD user.

In some instances, the pose of the view coordinate system VCS can be modified or move within the world coordinate system WCS. For example, the user may utilize control inputs on the HMD 200 and the techniques described herein to update the pose of the VCS at any time. In another example, an input from another source, such as the navigation system 20, could trigger a request to update, or an automatic update to the pose of the VCS. For example, the pose of the VCS could be set relative to a tracked anatomy. If the navigation system 20 detects movement of the tracked anatomy to an updated location, the HMD 200 can trigger a request to for the user to update, or automatically update, to the original pose of the VCS to move to an update pose relative to the updated location of the tracked anatomy. In another example, if the VCS is set relative to the surgical tool 22, the VCS pose can be continuously updated based on movement of the surgical tool 22.

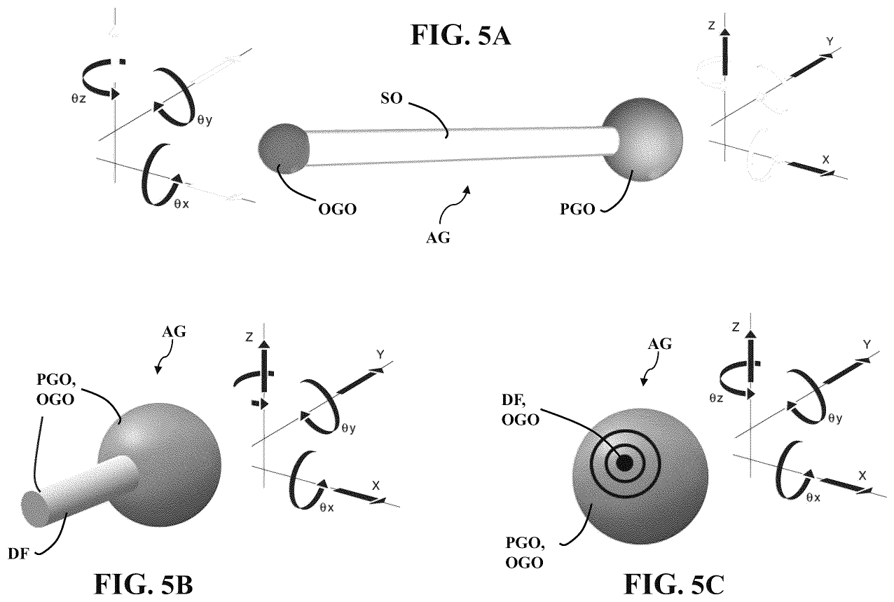

FIGS. 5A, 5B and 5C are illustrations of example alignment guides AG that can be displayed by the HMD 200 to assist a user to setting the pose of the view coordinate system VCS. The alignment guide AG can take many forms. The alignment guide AG is a 3D object that is computer-generated by the HMD controller 210 and presented on the HMD display 208 and onto the real-world view to enable the user of the HMD 200 to experience using the alignment guide AG relative to real-world environments and objects.

As shown in the example of FIG. 5A, the alignment guide AG can include a position guide object PGO dedicated to establishing a position for the view coordinate system VCS. The alignment guide AG can include an orientation guide object OGO dedicated to establishing an orientation for the view coordinate system VCS. The HMD controller 210 can receive control inputs from the sensing system 219 to enable selection and translational movement of the position guide object PGO to establish the position (within x, y, z planes) of the view coordinate system VCS. In FIG. 5A, the position guide object PGO and the orientation guide object OGO are separate objects that have separate functions. The HMD controller 210 can receive control inputs from the sensing system 219 to enable selection and rotational movement of the orientation guide object OGO to establish the orientation (pitch, yaw, roll) of the view coordinate system VCS.

In one implementation, as shown in FIG. 4A, the view coordinate system VCS has an origin (VCS-O) and the position guide object PGO is dedicated to establishing the position of the VCS origin and the orientation guide object OGO is dedicated to establishing the orientation of the view coordinate system VCS defined relative to the origin (VCS-O). In other words, user movement of the position guide object PGO causes translational movement of the VCS-O and user movement of the orientation guide object OGO causes rotation of the VCS coordinate system about the VCS-O.

The HMD controller 210 can utilize the gaze input to enable selection of the position guide object PGO and/or enable selection of the orientation guide object OGO. The gaze in can be a ‘look and stare’ input or dwell time input that looks for the eye to be staring for a threshold amount of time (e.g., 3 seconds). The control inputs can include a hand gesture input. The HMD controller 210 can utilize the hand gesture inputs to enable translational movement of the position guide object PGO and/or enable rotational movement of the orientation guide object OGO. The hand gesture input can be a finger pinch gesture for moving the alignment guide AG. The hand gesture can be detected within the view of the HMD 200 (e.g., so that the user looks at their hand on the HMD display 208) or can be detected while the hand is located outside of the view of the HMD 200. Other types of control inputs are contemplated for selecting and moving the alignment guide AG or any parts thereof. For example, voice commands through a microphone of the HMD 200 can be utilized.

As shown in the example of FIG. 5A, the position guide object PGO can be presented as a first volumetric object and the orientation guide object OGO can be presented as a second volumetric object. These volumetric objects can have sizes, shapes, or visual features that are the same as one another or different from one another. As shown in FIG. 5A, the first volumetric object and the second volumetric object are each a ball. Balls may be desirable as they provide a natural shape that is intuitive for grasping. However, other volumetric objects are considered, such as cubes, prisms, or any non-conventional or custom volumetric object. HMD 200 can present the first volumetric object with a first color or texture and/or present the second volumetric object with a second color different from the first color or texture. The different sizes, colors, textures of these objects PGO, OGO can help the user to clearly distinguish between the position and orientation control.

In FIG. 5A, the position guide object PGO is a larger volume than the orientation guide object OGO. The larger sized position guide object PGO may be desirable in situations when the alignment guide AG is virtually presented on the HMD display 208 in such a way that the position guide object PGO is virtually further away from the user's eyes and the orientation guide object OGO is virtually closer to the user's eyes. However, the opposite scenario is contemplated. Namely, the position guide object PGO may a smaller volume than the orientation guide object OGO and the alignment guide AG may be virtually presented on the HMD display 208 in such a way that the orientation guide object OGO is virtually further away from the user's eyes and the position guide object PGO is virtually closer to the user's eyes.

The HMD 200 can present the position guide object PGO as being spaced apart from the orientation guide object OGO. As shown in the example of FIG. 5A, a straight object SO can be virtually coupled between the position guide object PGO and the orientation guide object OGO. The straight object SO can be rigidly fixed to both the position guide object PGO and the orientation guide object OGO such that the straight object SO, the position guide object PGO, and the orientation guide object OGO collectively form a virtual rigid body. As a virtual rigid body, any movement to any of the objects PGO, OGO, SO, will cause a corresponding movement to the other objects. In other examples, the straight object SO may be fixed only to the orientation guide object OGO but pivotable relative to the position guide object PGO. The straight object SO can have a fixed length defined between the position guide object PGO and the orientation guide object OGO. The fixed length can spatially constrain the position guide object PGO and the orientation guide object OGO relative to one another. The fixed length can be predetermined to provide an object length that maximizes user-experience and is not excessively large or small. Alternatively, the straight object SO can have a variable length. The length can be varied by the user of the HMD 200 using configurable settings, or by the user stretching or contracting the length of the alignment guide AG, e.g., using control input and sensors 217. The straight object SO can be presented to provide an intuitive experience to help the HMD user understand the relationship between position and orientation. The length of the straight object SO helps create a visual line between the user's eyes and the desired orientation. The orientation guide object OGO and straight object SO can pivot 360 degrees around the position guide object PGO in any plane.

Alternatively, as shown in FIGS. 5B and 5C, the alignment guide AG can be an object that combines the functions of the position guide object PGO and the orientation guide object OGO into a simpler volumetric form than the three volumetric objects shown in FIG. 5A. In the examples of FIGS. 5B and 5C, the alignment guide AG can include one or more volumetric object(s) with a directional feature DF to indicate orientation. As shown, the volumetric object(s) include a sphere. Again, other types of volumes are contemplated.

In the example of FIG. 5B, the alignment guide AG includes two volumetric objects, i.e., a sphere and a directional feature DF extending from the sphere. The directional feature DF is the straight object SO extending from the sphere and being rigidly attached to the sphere. The straight object SO can alternatively be a directional arrow or virtual ray that extends from the sphere. In this example, the ball of the orientation guide object OGO from FIG. 5A is effectively eliminated and is substituted for the straight object SO. Here, the sphere and/or the straight object SO can be selected and translated and/or rotated to establish the pose of the view coordinate system VCS. For example, the sphere can be used to set the VCS position and the straight object SO can be used to set the VCS orientation. Thus, the sphere and the straight object SO can both function as the position guide object PGO and the orientation guide object OGO. Additionally, or alternatively, the sphere can function as one of the position guide object PGO or the orientation guide object OGO, while the straight object can function as the other.

In FIG. 5C, the alignment guide AG includes one volumetric object, i.e., a sphere and a directional feature DF on the sphere. The directional feature DF is an indicator placed on the surface of the sphere. Here, the indicator is a realized as a bullseye that is formed on the sphere surface. Of course, any type of indicator is possible, including a dot, or crosshair, or reticle. In this example, both the ball of the orientation guide object OGO and the straight object SO from FIG. 5A are effectively eliminated and substituted by the directional feature DF indicator. Here, the user need only to select the sphere. The user can translate the sphere in space to establish the VCS position and the user can rotate the sphere so that the bullseye aligns to the desired orientation for establishing the VCS orientation. Thus, the sphere can function as both the position guide object PGO and the orientation guide object OGO. Alternatively, the sphere can function as the position guide object PGO, while the directional feature DF can function as the orientation guide object OGO.

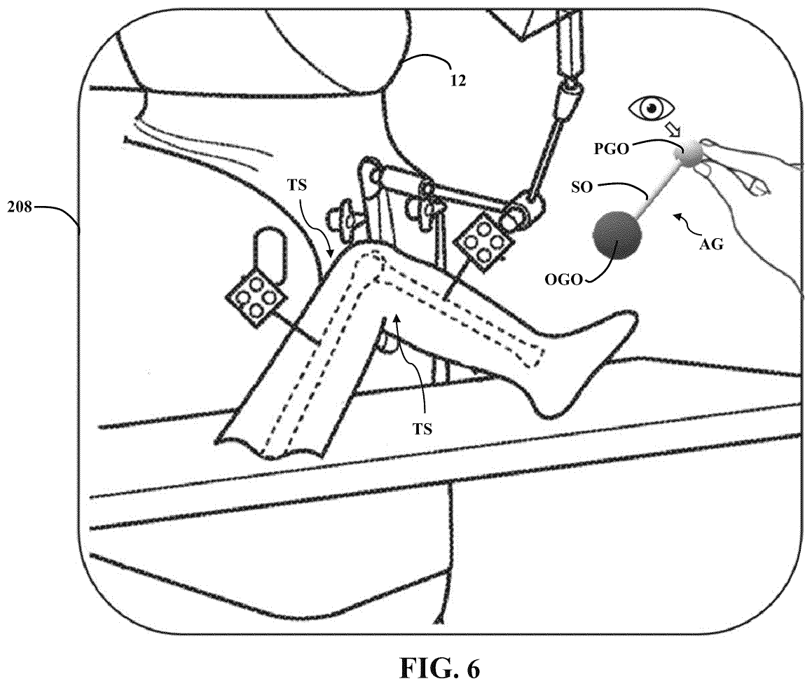

FIGS. 6-8 illustrate sample first-person views from or through the HMD display 208 wherein the user of the HMD 200 provides control inputs to select and manipulate the alignment guide AG for setting a pose of the view coordinate system VCS, according to one implementation. In these figures, the view on (or through) the HMD display 208 includes a partial real-world view of the surgical system 10 of FIG. 1, including a real-world view of the manipulator 11 and the target site TS of the patient on the surgical table. Of course, depending on where the user is looking, the object presented in the first-person view will be different. As described above, the real-world view may be implemented by a video stream reproducing the real-world view or by a transparent lens/visor or waveguide that enables the user to naturally see the real-world view. Here, the target site TS is the knee joint of the patient. However, the target site can be any anatomical joint, such as a hip joint, shoulder joint, ankle joint, or any part of the spine. Also, in FIGS. 6-8, one example of the alignment guide AG is shown, namely, the example of FIG. 5A. However, the alignment guide AG may be presented using any other implementation described herein. Additionally, the steps represented in FIGS. 6-8 involve establishment of the position of the view coordinate system VCS with the position guide object PGO prior to establishment of the orientation of the view coordinate system VCS with the orientation guide object OGO. However, the opposite may occur, and the steps may be different depending on the configuration of the alignment guide AG and/or how the user wishes to perform the calibration process. Furthermore, the alignment guide AG may have opacity or transparency that can be adjusted by the user of the HMD 200. When transparent, or semi-transparent, the real-world view can be seen through the alignment guide AG.

In FIG. 6A, the alignment guide AG is presented on the HMD display 208 to begin the calibration process for the view coordinate system VCS. This process may be initiated automatically or by the user-manually selecting an option on the HMD 200 to perform this process. Once the process begins, the alignment guide AG is presented as a computer-generated object combined or overlayed onto the real-world view. In this example, the position guide object PGO is virtually shown further away from the user's eyes and the orientation guide object OGO is virtually shown closer to the user's eyes. The user initially selects the position guide object PGO by providing control input to the sensing system 219. In this example, selection is made by staring at the position guide object PGO for a threshold time (indicated by the eye icon and arrow for simplicity). Once the position guide object is selected PGO, the HMD controller 210 may change the way the object looks, such as by changing its color, to indicate that the object has been selected. Having selected the position guide object PGO, the user can now provide additional control inputs to the sensing system 219 to translate the object. In this example, the user utilizes gesture control to virtually pinch the position guide object PGO in preparation for moving the same. In FIGS. 6-8, the user sees their hand (or a virtual representation of their hand) in the real-world view. However, the gesture control could be performed and detected outside of the HMD display 208 and the user may not see their hand or virtual representation of their hand.

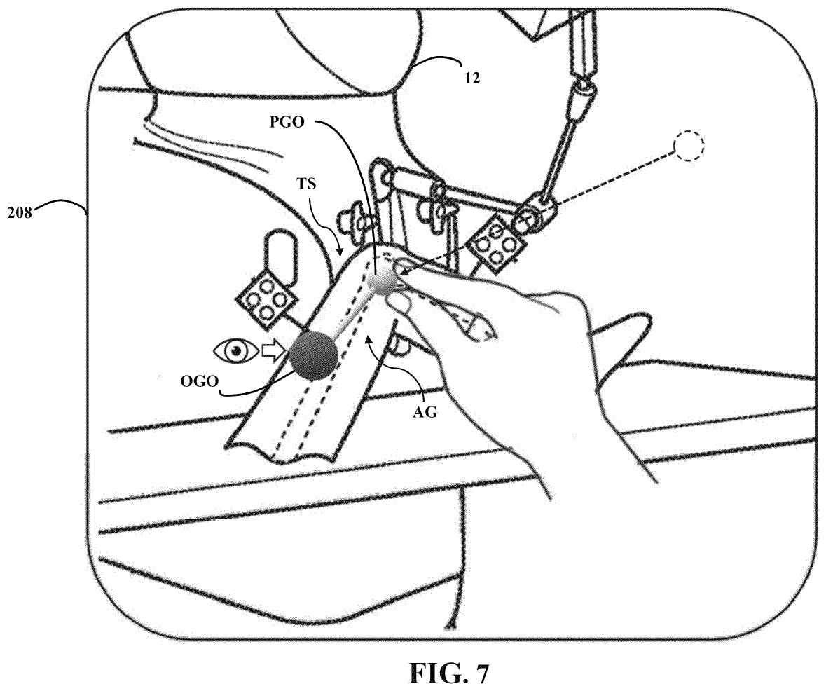

Continuing to FIG. 7, the user moves the position guide object PGO to the desired position to establish a position of the view coordinate system VCS. FIG. 7 illustrates movement of the position guide object PGO from its prior position in FIG. 6 (shown by dashed circle) to the current position. The direction of movement is indicated by a dashed line for illustrative purposes. In this example, the user locates the position guide object PGO to be directly above to the target site TS, and more specifically, directly in the middle of the knee joint. Notably, the user-defined location of the position guide object PGO will enable the view coordinate system VCS to be placed at position that is optimized for the surgeon's height and posture relative to the target site TS. The user can also move the position guide object PGO closer to or further away from the target site TS by moving the position guide object PGO towards their eyes or away from their eyes, respectively. During translation of the position guide object PGO, the straight object SO and orientation guide object OGO will correspondingly translate.

Once the position guide object PGO is set over the target site TS, the user can confirm the desired position of the VCS after this step. Alternatively, the user may wait until the moving the orientation guide object OGO to confirm the final pose of the VCS. Having placed the position guide object PGO in FIG. 7, the user subsequently selects the orientation guide object OGO by providing control input to the sensing system 219. In this example, selection is similarly made by staring at the orientation guide object OGO.

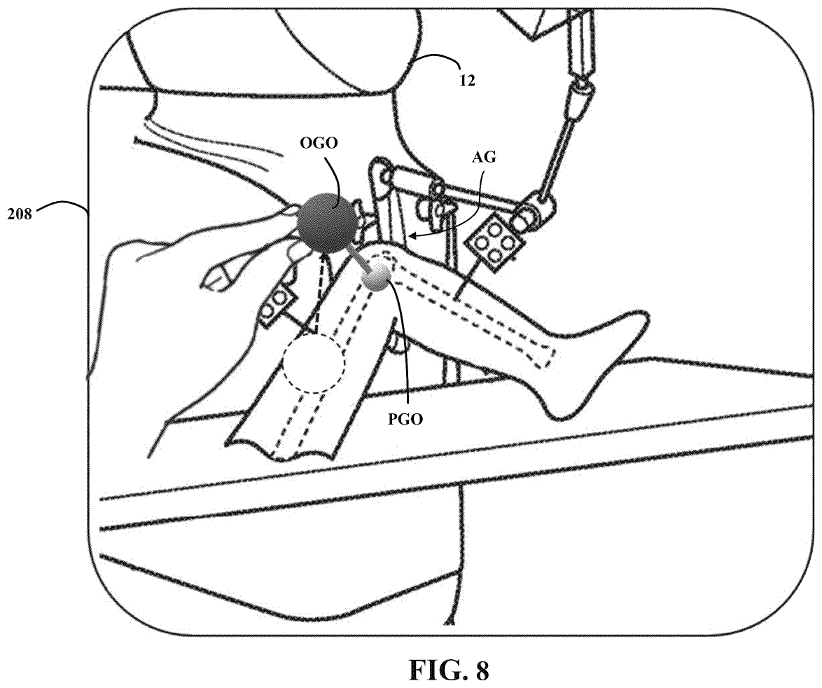

Continuing to FIG. 8, having selected the orientation guide object OGO, the user can now provide additional control inputs to the sensing system 219 to rotate the orientation guide object OGO. In this example, the user utilizes gesture control to virtually pinch the orientation guide object OGO for moving the same. FIG. 8 illustrates rotation of the orientation guide object OGO from its prior orientation in FIG. 7 (shown by dashed circle) to the current orientation. The direction of movement is indicated by a dashed line for illustrative purposes. During rotation of the orientation guide object OGO, the position guide object PGO remains in the prior established position. The straight object SO will correspondingly rotate with rotation of the orientation guide object OGO. The position guide object PGO may also correspondingly rotate with rotation of the orientation guide object OGO if the straight object SO is rigidly fixed to the position guide object PGO. Alternatively, the position guide object PGO may not correspondingly rotate with rotation of the orientation guide object OGO if the straight object SO is pivotable with respect to the position guide object PGO.

In FIG. 8, the user orients the orientation guide object OGO so that the straight object SO is substantially in-line with the user's sight of the target site TS. In other words, the user may point the alignment guide AG towards their eyes. As a result, the user-defined orientation of the alignment guide AG will enable the view coordinate system VCS to be oriented in a manner that is optimized for the surgeon's height and posture relative to the target site TS. Other manners of configuring the orientation of the alignment guide AG may be preferred by the user.

To further assist the user with visualizing the pose of the view coordinate system VCS, the HMD controller 210 can simultaneously present a representation of the view coordinate system VCS and the alignment guide AG on the HMD display 208. The representation of the view coordinate system VCS can be, for example, 2D or 3D grids, axes, or planes, which can be combined with or superimposed onto real-world views. The HMD controller 210 can translate the VCS representation in correspondence with translational movement of the position guide object PGO. The HMD controller 210 can rotate the VCS representation in correspondence with rotational movement of the orientation guide object OGO.

Having configured the position and orientation of the alignment guide AG, the user can confirm the final pose of the view coordinate system VCS. This confirmation may be performed automatically by the HMD controller 210 or may be confirmed by the use providing a control input to the HMD controller 210. The HMD controller 210 can save, in a non-transitory memory, the pose of the view coordinate system VCS established by the user. The VCS pose can be defined by the location of the position guide object PGO and the relative angle between the position guide object PGO and the orientation guide object OGO. The HMD controller 210 can retrieve, from the non-transitory memory, the established pose of the view coordinate system VCS during any subsequent use of the HMD by the user. This way, the user/surgeon need not re-calibrate the view coordinate system VCS before every procedure. Additionally, the established pose of the VCS can be associated with data identifying the type of procedure. For example, a specific surgeon may have one preferred VCS pose for a total knee procedure while having a different preferred VCS pose for a partial knee procedure. Additionally, multiple view coordinate systems VCS can be configured as described. The view coordinate systems VCS can be used to display the virtual objects VO in multiple ways. For example, the user could configure one VCS for one region or object (such as the femur) and a second VCS for another region or object (such as the tibia). Additionally, a dedicated VCS could be defined to present virtual objects VO relative to the manipulator 12. These multiple view coordinate systems VCS can be used simultaneously or at separate times during a procedure to present virtual objects VO within the respective VCS.

The saving and retrieving of the VCS settings can be based on a unique assignment of the HMD 200 to the user/surgeon or by the user/surgeon securely logging into the HMD 200. The VCS settings can be saved in local memory of the HMD 200, or on any other memory, including the memory of the navigation system, a memory of a connectivity system (CK) or remote server (RS) as shown in FIG. 1. Additionally, individual HMDs could each have their own establish view coordinate systems VCS settings associated/stored therewith. In other examples, the view coordinate systems VCS may be configured based on default setting associated with the host system/device with which the HMD 200 is operating in conjunction. For example, when the HMD 200 is used the navigation system 20, the HMD 200 may automatically load predetermined parameters of the VCS that are associated with the navigation system 20.

In one implementation, the alignment guide AG is not presented at the same time as the virtual object(s) VO. The virtual object(s) VO will later be presented within the view coordinate system VCS only after the VCS pose is configured using the alignment guide AG.

Alternatively, the alignment guide AG can be used specifically to orient the virtual object(s) VO, such as 3D object or virtual panel, as shown in FIG. 4B. The virtual object VO may, or may not, be displayed within the view coordinate system VCS. Here, the similar techniques described above with respect to using the alignment guide AG to set the pose of the VCS can be equally applied to setting to the pose of the virtual object VO. The HMD controller 210 can simultaneously present the virtual object VO and the alignment guide AG on the HMD display 208. The HMD controller 210 can translate the virtual object VO in correspondence with translational movement of the alignment guide AG or the position guide object PGO. The HMD controller 210 can rotate the virtual object VO in correspondence with rotational movement of the alignment guide AG or orientation guide object OGO. Hence, the alignment guide AG can serve as a tool to enable the user to easily set the pose of any object they may encounter on the HMD 200.

The surgeon/user has the option to selectively turn on/off any of the described features using the HMD 200. For example, the surgeon/user may select a virtual button or select an option from a virtual menu presented by the HMD 200 to activate the alignment guide AG, or to configure/modify a virtual object VO. The button could also be a physical button provided by the HMD 200 system, such as on the headset or on a hand-held controller. The controls further enable the surgeon/user to reset the alignment process AG if desired. Additionally, any controls described in the system 10 can be used to modify the presentation of any virtual object on the HMD 200. For example, buttons provided on a surgical tool 22 or probe tool can be used to selective turn on/off presentation of a virtual object VO on the HMD display 208. Additionally, whenever the HMD 200 detects an object and/or whenever a virtual object VO is presented, the HMD 200 can provide an audible alert through its speaker system or otherwise provide haptic (vibratory) feedback to the user. Furthermore, any of the techniques described herein that involve the HMD user/surgeon providing input to modify the alignment guide AG or any virtual object VO may alternatively be performed by another system (or HMD) to avoid distracting the user/surgeon during the procedure or to prevent the user/surgeon from using their hands. The other system or HMD can be coupled with the HMD 200 using a network or wired connection. In additional implementations, any parameters or setting involving the alignment guide AG or any virtual object VO may be pre-defined in the software of the HMD 200 or any related system. The user/surgeon may change selections or settings for the alignment guide AG or any virtual object VO, as desired, e.g., for each procedure. The HMD 200 or the relate system may monitor any changes to the settings during the procedure and save these settings for the next procedure.

### B. Intelligent Contextual Display of Virtual Objects on HMD

With reference to FIGS. 9-13, the concepts described herein further include the dynamic display of one or more virtual objects VO on the HMD display 208 at appropriate locations and at appropriate times to the user. In one example, the virtual object(s) VO are specifically presented relative to the user-calibrated pose of the view coordinate system VCS, as described above. However, the techniques described herein are not limited to such. Certain techniques may not involve displaying the virtual object(s) VO relative to any described coordinate system, such as the HMD coordinate system HMDCS.

As will be understood from the description herein, the described techniques provide an intuitive and selective configuration dictating how information or how much information is displayed to the user/surgeon of the HMD 200. The described techniques can display the information that the user/surgeon needs when they need such information and where they need such information to be displayed. In turn, the described techniques avoid displaying an overwhelming amount of information and avoid displaying information in undesirable location. Advantageously, the described techniques provide such benefits while minimizing impairment to the user/surgeon's view and minimizing disruption or distraction to the user/surgeon.

### 1. Virtual Objects

The one or more virtual objects VO are computer-generated and can be 2D or 3D objects or combinations thereof. The controller(s) can present, on the HMD display 208, the one or more virtual objects VO combined with, or overlaid onto, the real-world view.

The virtual objects VO can be a series of virtual objects VO that are presented one after the other. Multiple virtual objects VO can be simultaneously displayed. The virtual objects VO can include parent and sub-objects or nested virtual objects. The virtual object(s) VO can be translated and/or rotated by the user using the control inputs (such as gaze and gesture) and sensing system 219, as described above. The user can also move the virtual object(s) VO closer to or further away by moving the virtual object(s) VO towards their eyes or away from their eyes, respectively. In doing so, the size or the virtual object(s) VO may respectively increase or decrease in size depending on the magnitude and direction of the movement. The aspect ratio of virtual object(s) VO may remain the same during any such movements. Furthermore, any of the virtual object(s) VO described herein may have opacity or transparency that can be adjusted by the user of the HMD 200. When transparent, or semi-transparent, the real-world view can be seen through the virtual object VO. Certain real-world objects, such as the user's hand may take priority over any virtual object VO. Hence, when the hand is detected by the HMD 200, the hand will be displayed in front of the virtual object VO. A priority setting may be set for each individual virtual object VO. For example, virtual objects VO may have a criticality or importance setting. A more critical/important virtual object VO may be virtually displayed over a less critical/important virtual object VO and/or displayed closer to the user's eyes than a less critical/important virtual object VO. The surgeon/user may adjust the preferences as needed.

The virtual object(s) VO can display any information related or relevant to the surgeon, patient, or surgical procedure. The surgical information may, but need not, be related to the process of actually performing surgery. The surgical information can be pre-operative, intraoperative, or post-operative surgical information. Examples of surgical information include but are not limited to: patient information, medical images (e.g., CT scan or volume, X-rays, etc.), surgical guidance information (e.g., tool interaction with target site), surgical planning information, an anatomical model, an implant model, a cut plan, a resection plan or volume, a virtual boundary VB or cutting boundary, surgical tool information, operating room or tool setup information, surgical step information, clinical application information, surgical alerts, notifications or warnings, and the like. The surgical information can be a step of the surgical procedure. The step of the surgical procedure can include but are not limited to: a pre-operative planning step, an operating room setup step, an anatomical registration step, an intra-operative planning step, an anatomical preparation step, or a post-operative evaluation step. The virtual object(s) VO can be a 3D and/or 2D surgical object relevant to any of the above information. Examples of such virtual objects VO include but are not limited to: virtual screens, windows, or information panels, a 3D model of a bone, a 3D model of an implant, and a 3D surgical plan.

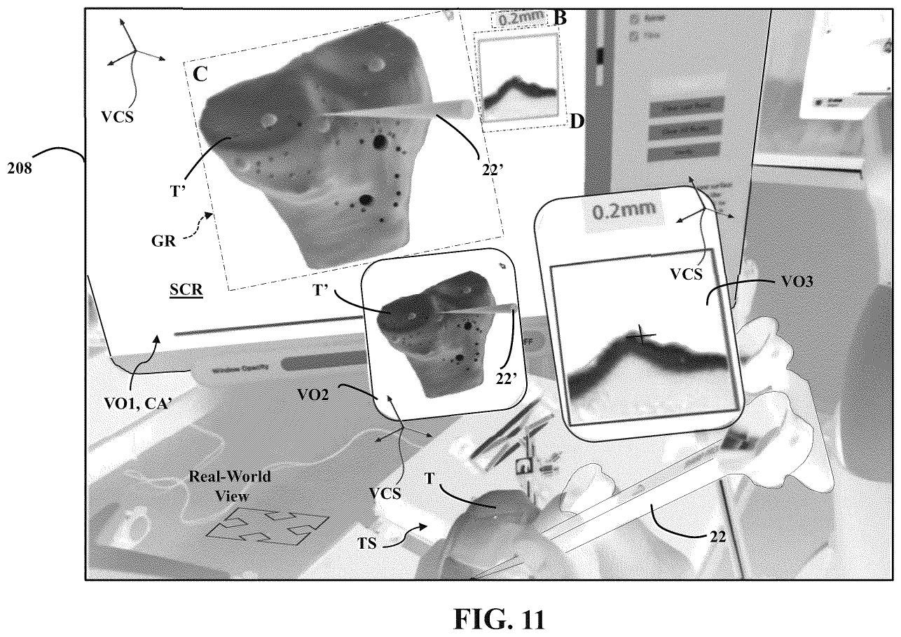

In one example, the virtual object(s) VO can be a virtual information panel VP, such as those shown in FIGS. 11-13. The virtual information panel VP can include 2D and/or 3D graphical elements, as well as text. The virtual information panel VP can be configured to present any relevant information in a “window” style format. The virtual information panel VP can have minimal thickness to emulate a flat object, such as a flat screen.

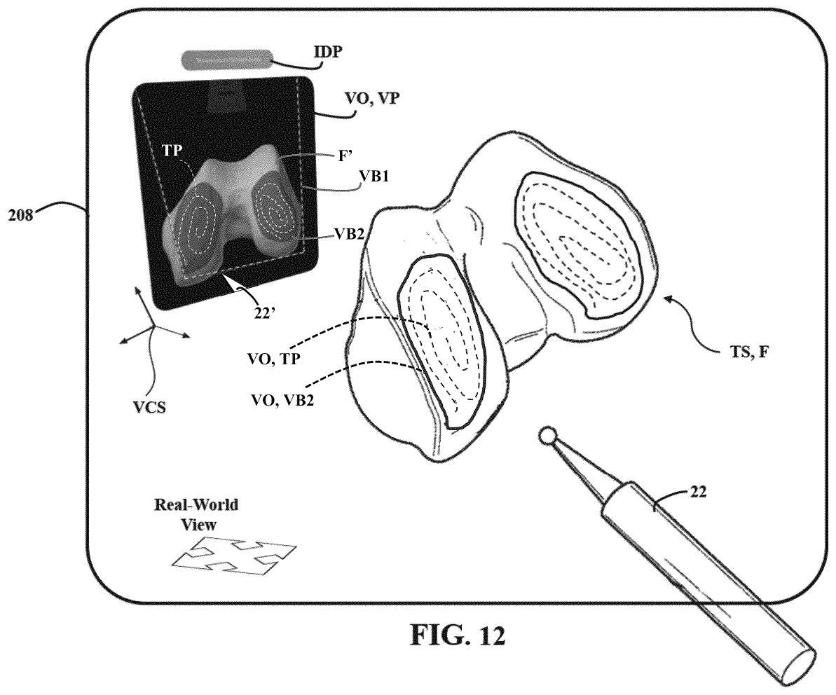

As shown in FIG. 12, the virtual information panel VP can include an identification panel IDP that can identify the contents of the virtual information panel VP. For example, in FIG. 12, the virtual information panel illustrates information relevant to a bone preparation process and the identification panel IDP identifies the ‘resection view. The identification panel IDP can be spaced above but constrained to movement of the virtual information panel VP, as shown. Alternatively, the identification panel IDP can be integrated into the panel VP or placed anywhere else relative the panel VP.

Specific examples of virtual objects VO and how they are display will be described in greater detail below.

### 2. Recognition of Surgical Information

In one implementation, the HMD 200 can automatically display one or more virtual object(s) VO specifically in response to a recognition of surgical information. The surgical information can be recognized according to various implementations and from various sources, as will be described below.

a. Obtaining Surgical Information from Video Stream Analysis of Host System/Device Software Application

In one example, the surgical information can be identified and/or extracted from the navigation system 20 or any host system/device (e.g., in the operating room) that is configured to display a software application, such as the clinical application CA presented by the navigation system 20. Although the navigation system 20 and its clinical application CA is largely described herein as one example, the host system/device 20 and respective software application can take various forms. For example, the host system/device 20 and software application can include any of: an endoscopic system that operates a software application for the endoscopic system; an imaging system (e.g., CT scanner) that operates a software application for the imaging system; a (CORE) console that operates a software application for operation of powered instruments; a surgical robot that operates a software application for controlling the surgical robot, a hand-held tool that operates a software application for controlling the hand-held tool, a surgical visualization system (e.g., arthroscope, ultrasound, laparoscope) that operates a software application for controlling the surgical visualization system, a surgical waste management system that operates a software application for controlling the surgical waste management system, a fluid management system that operates a software application for controlling the fluid management system, a sponge management system that operates a software application for controlling the sponge management system, a patient support apparatus that operates a software application for controlling the patient support apparatus, and the like.

Referring to FIGS. 1 and 2, the system 10 may include a connectivity system or kit, CS, which communicates between the navigation system 20 (or host system/device) and the HMD 200 to identify or extract such surgical information and perform other functions. In one example, the connectivity system CS includes a computing system (C), and an input device (ID) and output device (OD) and memory (M) coupled to the computing system C. The input device ID is configured to receive a video stream of the software/clinical application from the host system/device 20. For example, the connectivity system CS can receive a video stream of the clinical application CA from the navigation system 20. The input device ID can couple to the host system/device 20 using a wired input, such as a HMDI or DVI input. Conversion devices may be utilized to convert the format of the video stream (e.g., converting from DVI to HMDI for example). The computing system C is configured to automatically analyze and recognize information from the video stream of the software/clinical application. The computing system C may implement a stream analyzer SA to perform this function. In response to recognition of the information, the computing system C can generate the virtual object(s) VO related to the recognized information. The connectivity system CS can also include a communicator COM, which is configured to communicate with the HMD 200. The communicator COM can include any one or more devices that enable such communication. In one example, the communicator COM includes a wireless communication system, such as a WiFi router, Bluetooth transmitter, or the like. The HMD 200 is configured to communicate using the chosen communication method provided by the connectivity system CS. The output device OD may be the communicator COM itself, or the output device OD may be coupled to the communicator COM. The connectivity system CS may also be configured to receive any other type of data from the host system/device, such as control data, calibration data, or other information related to operation of the host system/device.

As shown in FIG. 1, the connectivity system CS can be a standalone device separate from the host system/device 20. The connectivity system CS can include a housing H. The housing H can form a device separate from the navigation system 20 or HMD 200. The housing H can store the various components of the connectivity system CS, including the computing system C and software, input device ID, memory M and communicator COM components. A mount MT can be attached to the housing H to enable the housing H to be mounted to a component of the host system/device 20, such as a display or a component of a movable cart of the host system/device 20. For example, the mount MT can include a mounting bracket to fix to a host component or a mounting hook to hang the housing H onto a display.

In other implementations, the connectivity system CS can be integrated, in part, or in whole, into the host system/device or navigation system 20. For example, the connectivity system CS can be implemented by the navigation controller 26 and the components of the connectivity system CS can be incorporated into the cart assembly 24. Also, the connectivity system CS can be integrated, in part, or in whole, into the HMD 200.

The connectivity system CS advantageously provides “plug and play” compatibility that surgeons and healthcare facilities demand. The connectivity system CS is well-adapted to be seamlessly compatible with existing surgical systems without significant re-development and re-design of the extended reality system and/or the surgical system. The connectivity system CS can be utilized to analyze the video stream provided by any host system/device provided by any manufacturer of surgical systems. The connectivity system CS can also communicate with any type of HMD that may be provided by any manufacturer of HMD systems The connectivity system CS provides information conversion capabilities between systems, even where such systems were not specifically developed to work together. In turn, the connectivity system CS can help ensure that the HMDs which are purchased by healthcare facilities or surgeons are compatible with the broad range of surgical systems and software required for various surgical procedures.

As described above, the surgical information can be obtained from a host device/system, such as the navigation system 20. In the example of the navigation system 20, the clinical application CA can be presented on one or more displays 28, 29 of the navigation system 20 (as shown in FIG. 1). Through connection to the connectivity system CS, the computing system C can obtain the video stream of the clinical application CA. In effect, the computing system C can obtain video, in real-time, corresponding to imagery, text or information is configured to be presented by the clinical application CA. The clinical application CA can be presented on the displays 28, 29 of the navigation system 20 while the video stream is obtained by the computing system C, and the video stream can reflect real-time user modifications to the clinical application CA. However, it is not always required that the clinical application CA be presented (e.g., if the display 28, 29 is turned off). The computing system C utilizes the stream analyzer SA to analyze and recognize the surgical information from the video stream of the clinical application CA. Notably, the techniques described herein may be performed by the navigation system 20 (or host system/device) in situations where the components of the connectivity system CS are integrated into the navigation system 20 (or host system/device) instead of being a stand-alone device. In such instances, the computing system C can be understood as including the navigation controller 26 and its respective components, or any components native to the host system/device.

In one implementation, the computing system C utilizes the stream analyzer SA to recognize the surgical information by automatically identifying text presented by the clinical application CA. The stream analyzer SA can utilize any text recognition algorithm to perform this function, such as optical character recognition OCR, visual text recognition, scene text recognition, natural language processing NLP, any combination thereof, and the like.

For any situation where text is detected, the computing system C can implement an algorithm to verify the accuracy of the extracted text. For example, the computing system C can cross-reference extracted text to database of dictionary words or expected words. Expected words can be specific words produced by the software/clinical application. The accuracy check can be character level or world level accuracy checks. If errors are detected, the computing system C can produce an error warning, e.g., on the HMD 200 or provide some other visual indication to the user of the HMD 200 to understand that the extracted text was not suitable for display. Text extraction errors can be collected in memory and/or processed (e.g., using machine learning) to reduce such errors in the future.

Additionally, or alternatively, the computing system C utilizes the stream analyzer SA to recognize the surgical information by automatically identifying imagery or graphics presented by the clinical application CA. The stream analyzer SA can utilize any image recognition algorithm to perform this function, such as segmentation, bounding boxes, pattern recognition, shape modeling, machine learning models, deep learning, neural networks, convolutional neural networks, any combination thereof, and the like. Additionally, or alternatively, the computing system C utilizes the stream analyzer SA to recognize the surgical information by automatically identifying user inputs provided on the clinical application. Such inputs may include mouse movements or behavior, cursor selections, inputted text (e.g., using a keyboard), screen selections, icon selections, movement, or manipulation of graphical objects, such as scroll bars, up/down arrows, bone models, implants, and the like.

The surgical information identified or extracted by the computing system C may include any information that may be relevant to the surgeon, patient, or surgical procedure. The surgical information may, but need not, be related to the process of actually performing surgery. The surgical information can be pre-operative surgical information. Alternatively, surgical information can include post-operative information, such as reports, etc. Examples of surgical information include but are not limited to: patient information, medical images (e.g., CT scan or volume, X-rays, etc.), surgical guidance information (e.g., tool interaction with target site), surgical planning information, an anatomical model, an implant model, a cut plan, a resection plan or volume, a virtual boundary VB or cutting boundary, surgical tool information, operating room or tool setup information, surgical step information, clinical application information, surgical alerts, notifications or warnings, and the like. The surgical information can be a step of the surgical procedure. The step of the surgical procedure can include but are not limited to: a pre-operative planning step, an operating room setup step, an anatomical registration step, an intra-operative planning step, an anatomical preparation step, or a post-operative evaluation step. The surgical information detected can include initialization, progression, or completion of any surgical step. The surgical information detected can include a time component or duration defining presence or absence of any of the described surgical information.

The information identified and/or extracted from the software/clinical application of the host system/device 20 depends on what the software/clinical application is configured to present. Referring to FIG. 9, for example, the clinical application can CA have a plurality of different screens SCR related to the surgical procedure. The screen SCR shown in FIG. 9 is a “Bone preparation” screen. Other screens SCR on the clinical application CA may include but are not limited to: “pre-op check,” “bone registration,” “intra-op planning,” “bone preparation” and “case completion.” Of course, the exact wording of the screen SCR may vary depending on the clinical application CA use case. Each screen SCR can have a screen identifier SI. Sometimes, the screen identifier can be a title of the screen, such as the “bone preparation” text provided at the top of FIG. 9. For example, when each title selected, e.g., using a mouse, the respective screen SCR for that title is shown on the clinical application CA. In one example, the computing system C can utilize the stream analyzer SA to automatically identify the screen identifier SI of the active one of the screens SCR of the clinical application CA. This can be performed by identifying the text of the title and/or by identifying any other graphic or text that can identify the contents of the screen SCR. For example, in FIG. 9, the stream analyzer SA can recognize that the “Bone Preparation” text located at “A”, which identifies the current screen in a scroll bar to switch between screens. A bounding box is illustrated to indicate identification of these screen identifiers SI. FIG. 10 presents another example, wherein the screen SCR is a “bone registration screen”. Again, the stream analyzer SA can identify the screen SCR based on detecting the screen identifiers SI, e.g., at the top of the screen SCR or by the scroll bar at the bottom of the screen SCR. The information that identifies the screen SCR can be located anywhere in the screen SCR. Alternatively, the stream analyzer SA can specifically monitor changes in information provided only within a specified region on the screen SCR. In another example, a detection region could be defined around a location on the screen SCR where an icon or a visual indicator is/would be displayed. The recognition of a change in pixel intensity or color in that detection region could indicate a triggered event.

Additionally, or alternatively, the computing system C can use the stream analyzer SA to identify or extract any information from the clinical application CA, regardless of identifying the specific screen SCR on which such information is presented. For example, the stream analyzer SA can detect certain text/graphics that may be unique to the particular screen SCR. In FIG. 9, for example, the stream analyzer SA may detect the word “tibia” (at B in the upper left-hand corner of the screen). The word “tibia” can be used by the computing system C to understand the context or contents of the screen SCR, e.g., that this screen involves bone preparation for the tibia (as compared to the femur, for example). The computing system C can have certain words stored in memory as being associated with the particular screens or steps of the procedure. Additionally, or alternatively, the specific graphics, such as presence of graphics of the bone, implant, registration spheres, the tool, or any object, could trigger the screen detection. For example, in FIG. 9, the stream analyzer SA may detect the model of the tibia bone, virtual boundary, and tool graphic, shown near the screen center (bound by box C). The computing system C can have certain graphics stored in memory as being associated with the particular screens or steps of the procedure. Here, the stream analyzer SA may use image/graphic recognition algorithm(s) to identify the displayed features so that the computing system C can determine the surgical context or screen contents.

In the example of FIG. 10, the stream analyzer SA can detect the model of the tibia bone, virtual (point registration) spheres on the surface of the tibia T′ model, and pointer 22′ graphic, shown near the screen center (bound by box C). This detect information can be used by the computing system C to understand the displayed context or contents, e.g., that the contents involve bone registration for the tibia (as compared to the bone preparation, for example).

FIG. 10 illustrates aspects of bone registration, which involves a process whereby the user touches certain points of the bone (e.g., tibia T) with a tracked probe tool 22 in order to register the pose of the bone to the navigation system 20. The probe tool 22 is used to physically touch the actual bone surface. In FIG. 10, the clinical application CA displays the real-time interaction between a graphical representation of the probe tool 22′ and the tibia bone T′. To guide the surgeon, the clinical application CA can show a real-time distance between the tip of the tool 22 and the bone T (e.g., 0.4 mm—shown at B in FIG. 10). The clinical application CA further can display a sub-window (at D in FIG. 10) that shows a patient image, such as a CT slice that can depict a real-time location of the tool tip relative to the bone structure in the image, such as the cortical bone. The sub-window can help the user further pinpoint the proper placement of the probe tool 22 relative to actual bone structure.

In some implementations, certain regions of the screen SCR can be monitored from the video stream to detect surgical information that subsequently triggers monitoring or detection of another region of the screen SCR. For example, the detection of specific text on the screen SCR can trigger clipping or reproduction of a graphical part of the screen SCR for display on the virtual object(s) VO, such as a virtual panel. To illustrate, in FIG. 9, the detection of the “bone preparation” screen identifier SI can trigger clipping and reproduction of the guidance region GR (inside box C) for display on the virtual object(s) VO. Similarly, in FIG. 10, the detection of the “bone registration” screen identifier SI can trigger clipping and reproduction of the guidance region GR (box C) for display on the virtual object(s) VO. Also, the detection of the word “distance” on the screen SCR (box B) can trigger the clipping and reproduction of the sub-window at (inside Box D) for display on the virtual object(s) VO. In some cases, the detected information may also be extracted or reproduced for display on the virtual object(s) VO. Alternatively, the detection of the information may be performed only for the purpose of triggering clipping or reproduction of another region the screen SCR. Bounding boxes or detection regions can be used to define boundaries of the region to clip or reproduce for presentation on virtual objects VO. The dimensions and parameters of the boxes or regions can be defined in any suitable way relative to the software application, such as using a pixel coordinate system, or the like.

Additionally, it is contemplated that any surgical information detected from any source can trigger clipping or reproduction of a region from any other source for presentation on the virtual object VO. The detected surgical information need not necessarily come from a screen SCR. Surgical information detected by any camera source described herein could trigger clipping or reproduction of a graphical object on a screen SCR for presentation of the virtual object(s) VO. Additionally, the computing system C can receive multiple video streams from multiple host systems/devices. For example, the navigation system 20 may detect a tracked surgical object using the localizer 34, which can then trigger the clipping or reproduction of a guidance region GR displayed on the clinical application CA. In another example, surgical information detected by an endoscopic camera may trigger the clipping or reproduction of endoscopic tool information displayed on a surgical system screen. In yet another example, an imaging system (e.g., X-ray scanner) may include a display that presents a software application for the imaging system. The surgical information can be detected from a video stream of the imaging system software application and this surgical information can trigger the clipping or reproduction of a region displayed on the clinical application CA of the navigation system 20. In another example, a warning issued by the manipulator 12 can be detected and used to trigger clipping of textual information related to the warning from a video stream of surgical system. Numerous other possibilities are contemplated in view of the techniques described herein.

By monitoring certain regions of the software application and/or clipping or reproducing certain portions of the software application, the described techniques advantageously improve performance of the system, speed of processing information, and recognition accuracy. Namely, by monitoring certain regions for information, the computing system C need not waste resources on monitoring the entirety of the video stream contents and can utilize its processing power for other purposes. Additionally, by clipping or reproducing only certain regions of the software application for presentation on virtual object(s), the system can process and provide such information for display to the HMD 200 much faster than if the entire software screen were to be reproduced. Hence, these techniques further reduce the latency in virtual object presentation on the HMD 200.

In the numerous examples described above, the computing system C can be configured to monitor specific regions of the clinical application CA, such as those regions that are most likely to contain relevant information. The stream analyzer SA can monitor pixel activity, such as color, intensity, or pixel group movement, or pixel by pixel changes of sequential frames, etc. Additionally, the stream analyzer SA can monitor the video data to determine when certain information becomes present or absent. The presence or absence of information may trigger higher order determinations beyond mere presence/absence of information. Namely, the computing system C may be configured to infer surgical procedure context. For instance, the computing system C may infer that the bone registration process is completed by detecting absence of the pointer tool 22 for a threshold time, or by detecting presence of a window that indicates “registration is complete”.

While the information detected by the stream analyzer SA is shown in FIGS. 9 and 10, relative to the screen view, these figures are shown simply for illustrative purposes and are not intended to limit the function of the stream analyzer SA or how surgical information from the screens is obtained. It is understood that the stream analyzer SA can process the video stream of the software/clinical application with or without any graphical representation (as shown) and can process this information strictly within the computing system C, and unbeknownst to the user.

As will be described in detail below, certain virtual objects VO can be sub-objects, or sub-panels that display certain portions of information from other (parent) virtual objects or panels. These sub-panels can be presented to help the user/surgeon see smaller/detailed regions more clearly, e.g., by specifically displaying the zoomed in region. To facilitate this process, the stream analyzer SA can detect displayed information that is relevant to this sub-information. For example, in FIG. 10, the stream analyzer SA further detects the word “distance” and the displayed contents of the sub-window (at D). The stream analyzer SA can identify or extract this information in real-time for later presentation of virtual objects VO that can help the surgeon perform bone registration.

Other methods of obtaining surgical information from the host system/device software/clinical application are contemplated. Any of the techniques described above can be used individually or in combination.

b. Obtaining Surgical Information from Camera or Other Source(s)

The example surgical system 10 described has various camera sources. These camera sources can include the HMD camera 214 and the camera (visible light or machine vision camera) of the navigation system 20. Additional camera sources may include a camera source attached to the tool 22. Such tool cameras include, but are not limited to: a scope, an endoscope, a laparoscope, an arthroscope, and a microscope. Other camera sources may be in the operating room, such as other HMDs 200, a camera attached to the manipulator 12, or a dedicated (standalone) camera utilized for viewing a separate display device. Any of these cameras is a camera source for purposes of this description.

Any camera source can detect surgical information. Such surgical information may be detected at the target site TS or elsewhere. The surgical information detected by the camera source can be processed by any suitable controller or computing system, depending on the system configuration. Such controllers/computing systems can include but are not limited to, the camera controller 42, the navigation controller 26, the computing system C, manipulator controller 54, tool controller, or the HMD controller 210.

Surgical information detectable by the camera source may include any information that may be relevant to the surgeon, patient, or surgical procedure. The surgical information may, but need not, be related to the process of actually performing surgery. The surgical information can be pre-operative or post-operative surgical information. Examples of surgical information detectable by the camera source include but are not limited to: location and/or detection of any surgical object (such as the bone, tracker, tool, robot or end effector, sensitive tissues, retractors, surgical table, imaging device, etc.), tool identification, anatomy information, surgical guidance information (e.g., tool interaction with target site), interaction between tools, amount of bone removed or needed to be removed, tool path TP, tool calibration, tool or component installation, surgical planning information, identification of an obstruction to a tool, line-of sight obstructions, surgeon ergonomics or posture, and the like.

The surgical information detectable by the camera can be procedure information or a step of the surgical procedure. The step of the surgical procedure can include but are not limited to: a pre-operative planning step, an operating room setup step, an anatomical registration step, an intra-operative planning step, an anatomical preparation step, or a post-operative evaluation step. The surgical information detected can include initialization, progression, or completion of any surgical step. The surgical information detected can include a time component or duration defining presence or absence of any of the described surgical information.

The surgical information detectable by the camera source can also include any information presented by the clinical application CA on the navigation system 20 display 28, 29. For example, by the HMD user looking at the display 28, 29, the HMD camera 214 can capture a live-video stream of the clinical application CA. In so doing, the HMD camera 214 can detect the content of the clinical application CA on the displays 28, 29 and identify or extract surgical information. The HMD 200 can analyze the contents of the video stream in manner similar to that described above (e.g., by identifying or extracting text and/or images). The HMD 200 can perform this function using the HMD controller 210 and/or the computing system C and stream analyzer SA.

In another example, a camera source may be directed at a piece of equipment (e.g., such as a display or a control panel) to identify changes presented by the equipment to detect the surgical information. At the beginning of, or during the procedure the user/surgeon may select or define a region of the equipment to monitor for changes. The user/surgeon can also specify what information and when such information detected from the equipment should be presented on the HMD display 208.

Surgical information can also be detectable by other sensing features of the HMD 200, including any component of the sensing system 219 of the HMD 200, including the microphone, the IMUs 216, the tracking sensors 212, and/or the control input sensors 217. For example, the microphone may detect an alert, sound, or message outputted by the navigation system 20, such as a chime to indicate that a step has been successfully performed. The HMD 200 microphone can detect this chime and process the chime as input surgical information. Other methods of obtaining surgical information from such other sources are contemplated. Any of the techniques described above can be used individually or in combination.

### 2. Virtual Object Presentation

As described above, surgical information can be identified or extracted in various forms and by various systems, such as by analyzing the video stream of the software/clinical application and/or by obtaining input from any other source(s) such as cameras. The surgical information can be processed by any suitable controller or computing system, depending on the system configuration. Such controllers/computing systems can include but are not limited to, the camera controller 42, the navigation controller 26, the connectivity system CS, the computing system C, manipulator controller 54, tool controller, or the HMD controller 210. Whatever the applicable system used to process the surgical information is referred to in this section as the “controller(s).”

Upon processing of the surgical information, video data and/or command signals are communicated by the controller(s) to the HMD 200 for presentation. The HMD 200 can automatically display or be commanded to display, one or more virtual object(s) VO specifically in response to recognition of such surgical information. The software/clinical application may or may not be presented on the host system/device display concurrently while the virtual object VO is presented on the HMD display 208.

In one example, in response to recognition of surgical information, the virtual object(s) VO are specifically presented relative to the user-calibrated pose of the view coordinate system VCS and combined with the real-world view. The virtual object(s) VO can also be displayed at predetermined positions and/or orientations within the view coordinate system VCS, as described above. The predetermined position and orientation of the one or more virtual objects VO within the view coordinate system VCS can be based on: a default position and orientation; or a user-defined position and orientation. In one example, the virtual object VO and the predetermined pose are based on the recognized surgical information.

Alternatively, in response to recognition of surgical information, the virtual object(s) VO may be displayed relative to any described coordinate system, such as the HMD coordinate system HMDCS. In such instances, the virtual object(s) VO are also combined with the real-world view and can also be displayed at predetermined positions and/or orientations within the respective coordinate system, e.g., HMDCS.

The surgical information can be provided by the software/clinical application in a first format, e.g., that is native to the application. In response to recognition of the surgical information, the controller(s) can transform the surgical information from the software/clinical application into a second format adapted for presentation of the virtual object VO on the HMD 200. For example, the controller(s) can transform the surgical information into the second format by automatically performing one or more of the following: re-arranging or repositioning the surgical information; cropping or clipping the surgical information; and/or re-sizing the surgical information. Additionally, the controller(s) can graphically change a 2D object from the software/clinical application into a 3D object on the HMD 200. For example, the 3D object, such as a tool or bone, can appear to be extending out of the plane of the virtual panel VP. The controller(s) can add surface textures or perform color, filtering, opacity, aspect ratio and/or resolution modifications to the graphic information from the software/clinical application to improve appearance on the HMD display 208.

Furthermore, the controller(s) can duplicate a portion or an entirety of the video stream of the software/clinical application for display on the HMD 200 as the virtual object VO or virtual information panel VP. This duplication replicates the real-time presentation of the software/clinical application or portion thereof. For example, the controller(s) can duplicate a navigation guidance region GR on the virtual information panel VP. The navigation guidance region GR can display one or more surgical objects tracked by a localizer 34 of a navigation system 20. The controller(s) can also present, on the HMD display 208, a primary virtual panel VP displaying an entirety of software/clinical application combined with the real-world view while further simultaneously displaying a second virtual panel VP that includes information from, and/or portions of the software/clinical application.

FIGS. 11-13 further illustrate practical examples of virtual objects VO that can be displayed on the HMD display 208. These examples are provided merely for illustrative purposes. The techniques described herein can perform countless other examples involving the HMD 200 and display of virtual objects VO.

FIG. 11 illustrates a sample first-person view through the HMD display 208 wherein the HMD displays several virtual information panels VP over a real-world view of a target site TS. The surgical step in FIG. 11 involves bone registration. The example of FIG. 11 can be understood in the context of FIG. 10 and the description related to obtaining surgical information from the “bone registration” screen SCR of the clinical application CA. In FIG. 11, the HMD display 208 provides a real-world view of the tibia T, the pointer tool 22 used for touching the tibia T, the user's actual hand holding the tool 22, as well as the surrounding real-world environment. In this example, three separate virtual objects are presented, i.e., VO1, VO2, VO3. For purposes of this example, we understand that the virtual objects VO1, VO2, VO3 are being positioned in the view coordinate system VCS after configuration of the VCS using the alignment guide AG, and techniques described above. Specifically, in this example, the VCS position and orientation are established directly atop of the knee joint using the alignment guide AG. In this example, each of the virtual objects VO1, VO2, VO3 are combined into a live stream of the real-world view, as acquired by the camera 214 of the HMD 200. Each of the virtual objects VO1, VO2, VO3 are more specifically presented as virtual panels VP.

The first panel VO1 is a primary control window that replicates, in full, a real-time video stream from the clinical application CA. In this case, the first virtual panel VO1 fully replicates the full “bone registration” screen SCR of the clinical application CA, just as it is displayed on the clinical application CA (e.g., as shown in FIG. 10). Accordingly, the first panel VO1 can provide a quick and convenient way for the HMD user to access the clinical application CA in its entirety. Again, the clinical application CA may or may not be presented on the host system/device display 28, 29 concurrently while the first virtual panel VO1 is presented on the HMD display 208.

The first panel VO1 is presented at a location that is behind the target site TS. The first panel VO1 is also presented at predetermined pose within the view coordinate system VCS and defined and/or fixed in the world coordinate system WCS such that the first panel VO1 is conveniently optimized to enable the surgeon to easily see the full contents of the clinical application CA without obstructing the target site TS view.

Along with displaying the full clinical application CA, the first panel VO1 displays the navigation guidance region GR of the bone registration screen SCR wherein the guidance region GR displays a graphical representation of the pointer tool 22′ relative to a graphical representation of the tibia T′, as well as the relative relationship between these objects, as detected by the navigation system 20. Hence, as the user physically moves the real pointer tool 22 with their hand, the pose of the graphical representation of the pointer tool 22′ will change in real-time on the first panel VO1.

The bone registration screen SCR (or any screen) of the clinical application CA may be presented on the first panel VO1 in numerous ways. First, the user can select the bone registration screen SCR on using the control inputs (e.g., mouse) on the clinical application CA displayed using the navigation system 20. In response to selection of the screen SCR, the controller(s) can automatically detect the screen identifies SI indicating “bone registration” by analyzing the video stream, as shown, and described with respect to FIG. 10. Second, the HMD sensing system 219 can detect any user input for selecting the bone registration screen SCR on the first panel VO1, itself. As described, for example, the user input can be gaze and/or hand gesture based. In another example, the navigation system 20 can detect movement of the pointer tool 22. This information can be fed to the controller(s) to detect that bone registration is desired. In response, the controller(s) can automatically present the bone registration screen SCR. Several other ways are contemplated for automatically presenting a screen on the first panel VO1.

The second panel VO2 is also sub-view of the first panel VO1. More specifically, the second panel VO2 is dedicated to presenting a magnified view of the navigation guidance region GR, including graphical representations of the pointer tool 22′ and tibia T′. Hence, as the user physically moves the real pointer tool 22 with their hand, the pose of the graphical representation of the pointer tool 22′ will also change in real-time on the second panel VO2.

The controller(s) can automatically present the second panel VO2 is response to detection of various inputs. In one example, detecting the bone registration screen SCR can cause the HMD 200 to automatically display the second panel VO2. In another example, the navigation system 20 can detect movement of the pointer tool 22 or the touching of the tool 22 on the bone T to cause the second panel VO2 to be automatically displayed. In another example, the stream analyzer SA can detect movement of the graphical representation of the tool 22′ from the clinical application CA video stream. Several other ways are contemplated for automatically displaying such a sub-view.

To present the sub-contents on the second panel VO2, the controller(s) can crop or clip the contents from, and/or target a specific portion of, the clinical application CA video stream. For example, in response to detection of the “bone registration” screen identifier SI on the clinical application CA (FIG. 10), the controller(s) can immediately trigger the clipping and/or reproduction of the guidance region GR (box C). Video stream data within this detection region can be prioritized or treated separately from the video stream data used to display the contents on the first panel VO1, for example. In other examples, the camera 214 of the HMD 200 can detect the full contents of the first panel VO1 from the video stream of the camera 214 and extract the sub-portions of the first panel VO1 (e.g., the navigation guidance region GR) for obtaining the contents to display on the second panel VO2. The HMD controller 210 can apply similar cropping, clipping, reproduction and/or detection box techniques as described.

The second panel VO2 is presented at a location that is virtually closer to the user's eyes than the first panel VO1. The second panel VO2 is also presented at predetermined pose within the view coordinate system VCS and defined and/or fixed in the world coordinate system WCS such that the second panel VO2 is conveniently optimized to enable the surgeon to easily monitor the bone registration process in detail, and without obstructing the surgeons view of the target site TS.

The third panel VO3 is another sub-view of the first panel VO1. More specifically, the third panel VO3 is dedicated to presenting another portion of the bone registration screen SCR from the clinical application CA. Namely, the third panel VO3 presents a magnified view of the sub-window (at D in FIGS. 10 and 11) that shows a CT slice that depicts a real-time location of the tool 22 tip relative to the bone structure in the image, such as the cortical bone. Hence, as the user physically moves the real pointer tool 22 with their hand, the position of the crosshair of the pointer tool 22′ will also change in real-time on the third panel VO3. The third panel VO3 sub-window can help the surgeon specifically pinpoint the proper placement of the probe tool 22 relative to actual bone structure. The third panel VO3 also displays, a real-time computed distance between the tip of the tool 22 and the bone T (e.g., 0.2 mm—shown at B in FIGS. 10 and 11). The information presented on the third panel VO3 can be created from the video stream of the clinical application CA using any of the techniques above. Video stream data within this detection region can be prioritized or treated separately from the video stream data used to display the contents on the first panel VO1, for example.

The controller(s) can automatically present the contents of the third panel VO3 is response to detection of various inputs. In one example, the steam analyzer SA detects the word “distance” at (box B) appearing on the clinical application CA and in response the controller(s) trigger the clipping of the sub-window D for reproduction on the third panel VO3. The stream analyzer SA could also detect a specific distance or range of distances of the tool tip displayed at D, to automatically trigger presentation of the specific distance and/or the sub-window D on the third panel VO3. The distance or range of distances can be a threshold distance, for example. Also, the navigation system 20 can detect movement of the pointer tool 22 or the touching of the tool 22 on the bone T to cause the third panel VO3 to be automatically displayed. In another example, the stream analyzer SA can detect movement of the graphical representation of the tool 22′ from the clinical application CA video stream. Several other ways are contemplated for automatically displaying such a sub-view.

In another example, the camera 214 of the HMD 200 can detect the full contents of the first panel VO1 from the video stream of the camera 214 and extract the sub-portions of the first panel VO1 (e.g., regions B and D) for obtaining the contents to display on the third panel VO3. The HMD controller 210 can apply similar cropping, clipping, and/or detection box techniques as described.

Like the second panel VO2, the third panel VO3 is also presented at a location that is virtually closer to the user's eyes than the first panel VO1. The third panel VO3 can be presented side-by-side next to the second panel VO2, or at a location that is virtually closer to the user's eyes than the second panel VO2. The third panel VO3 is also presented at predetermined pose within the view coordinate system VCS and defined and/or fixed in the world coordinate system WCS such that the third panel VO3 is conveniently optimized to enable the surgeon to easily monitor the bone registration process in detail, and without obstructing the surgeons view of the target site TS.

Conveniently, the controller(s) deliberately omits certain information in the sub-contents on the second and third panels VO2, VO3 that otherwise are presented on the clinical application CA. For example, the second and third panels VO2, VO3 do not show scroll bars, screen titles, icons, or other clinical information provided by the clinical application CA. This design choice enables the surgeon to see only the information that is relevant to the step at hand, without overwhelming the surgeon with unnecessary information thereby cluttering the view and defeating the purpose of the sub-panel. Additionally, these techniques significantly reduce latency in producing the virtual objects VO on the HMD display 208. However, in some cases, the surgeon can customize specifically what is shown on the any virtual object VO based on their surgical preferences. For example, the second panel VO2 could be set to also show a count of how many registration points have been selected using the tracked probe tool 22. Also, the third panel VO3 could be set to also show the word “distance”.

Moreover, by simultaneously presenting the second and third panels VO2, VO3 in front of the first panel VO1, the surgeon can also see information presented on the clinical application CA that is otherwise not presented on the third panel VO3. For example, a warning may be displayed on the clinical application CA presented by the first panel VO1 that could affect how the surgeon performs the surgical step using the second and third panels VO2, VO3. This warning could be a registration accuracy warning, for instance.

In another example, the third panel VO3 could be a sub-view of the second panel VO2, e.g., in a nesting fashion. For example, the third panel VO3 could instead show a “zoomed in” or magnified view of the pointer tool 22′ and tibia T′ from the second panel VO2, whereby the surgeon may a magnified view of where the tip of the tool 22′ is relative to the registration spheres on the tibia T′ surface. In such instances, the second panel VO2 could remain displayed or temporarily disappear until it is detected (e.g., from stream analysis) that such a magnified view is no longer needed. Alternatively, instead of the third panel VO2, the second panel VO2 may automatically change to temporarily show the magnified view.

The techniques described above and shown in FIG. 11 relate to how the system may be utilized and the how the panels VO1, VO2, VO3 are displayed for the bone registration process. However, similar techniques can be utilized for any surgical procedure, for any surgical step, and for any screen SCR presented by the clinical application CA.

For instance, the techniques can be similarly applied during the bone preparation process. The first panel VO1 may similarly display the primary control window that replicates, in full, a real-time video stream of the “bone preparation” screen SCR of the clinical application CA (e.g., as shown in FIG. 9). In this example, the procedure is a total knee arthroplasty TKA. The second panel VO2 can also be displayed as a sub-view of the first panel VO1 and can be dedicated to presenting the navigation guidance region GR from the bone preparation screen SCR, including graphical representations of a cutting tool 22′ (such as a saw blade) and tibia T′, as well as the virtual cutting boundary VB. As the user physically moves the cutting tool 22 with their hand, the pose of the graphical representation of the cutting tool 22′ changes in real-time relative to the tibia T′ on the second panel VO2. To present the sub-contents on the second panel VO2, the controller(s) can crop and/or clip the contents from, and/or target a specific portion of, the detection box (C in FIG. 9), which specifically focuses on the portion of the bone preparation screen SCR that includes the navigation guidance region GR. The guidance region GR can be clipped for reproduction in response to detection of the screen identifier SI, i.e., “bone preparation” in FIG. 9. The third panel VO3, if utilized, could present a magnified interaction between the cutting tool 22′ and the virtual cutting boundary VB, for example, when the distance between the two reaches a threshold distance. In another example, the third panel VO3 may temporarily and automatically be presented for displaying a warning detected from the bone preparation screen SCR, such as a warning involving cutting accuracy of the tool 22.

FIG. 12 illustrates another example of using the system for bone preparation, specifically for partial knee arthroplasty PKA. FIG. 12 is a sample first-person view through the HMD display 208 wherein the HMD 200 displays the virtual object VO as the virtual panel VP at a specific pose within the view coordinate system VCS in response to detection of surgical information related to bone preparation. The virtual panel VP displays relevant surgical information to guide the user in preparing the femur F according to a surgical plan. The virtual panel VP displays a graphical representations of the femur F′ and tool 22′ as they move relative to each other. The virtual panel VP also displays virtual boundaries VB1, VB2 and a tool path TP. The virtual boundaries include a first boundary VB1, which is “keep in zone” which the tool 22 movement should not exceed. A second boundary VB2 can define the region to be removed from the femur F by the tool 22 in preparation for receiving the implants. The tool path TP can be a planned path along with the tool 22 traverses to remove the material within the virtual boundary VB2. Other surgical planning elements can be displayed other than those shown. In this example, the surgical information described above are also displayed as virtual object VO directly onto the femur F in the real-world view. Hence, the surgeon can see, on/through the HMD display 208, virtual boundary VB and tool path TP directly overlaid onto the femur F.

Accordingly, it is also contemplated that any virtual object(s) VO can additionally or alternatively be registered to the target site TS. Registration of the virtual objects VO to the target site TS can occur in numerous ways. In one example, registration can occur by registering the HMD 200 to the localizer coordinate system LCLZ. The registration device 220 (with corresponding HMD camera detectable elements and localizer detectable elements) can be used register the HMD 200 to the LCLZ. In another example, the HMD tracker 218 can be detected by the localizer 34. In yet another example, the HMD 200 can use its camera 214 to detect and continually track the target site TS (without the LCLZ tracking the HMD). The view of the registered virtual objects VO can be in the HMD coordinate system HMDCS, the world coordinate system WCS, or any suitable coordinate system.

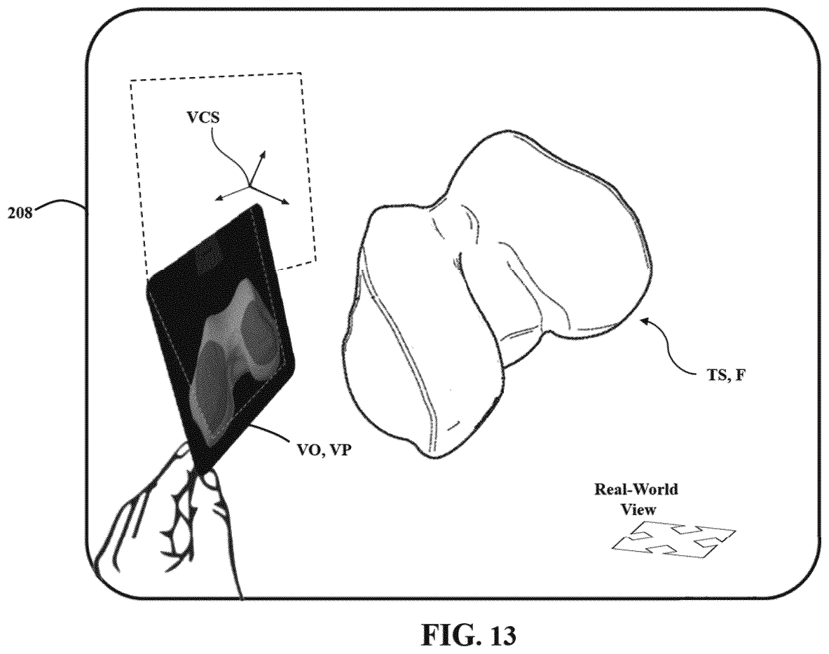

FIG. 13 illustrates another sample first-person view through the HMD display 208 wherein the HMD user provides control inputs to change a pose of the virtual panel VP of FIG. 12, according to one implementation. As established, the virtual object(s) VO, VP can be displayed at specific positions and orientations within the view coordinate system VCS. However, the user/surgeon may wish to adjust the pose of any virtual object VO. Accordingly, as shown in FIG. 13, the user/surgeon is utilizing gesture inputs to change the pose of the virtual panel VP from the prior pose (dotted line) to an updated pose defined within the VCS. The updated pose and context of the virtual object VO can be saved by the controller(s) for later retrieval. As such, when the user/surgeon performs a similar step in a subsequent procedure, the virtual object VO can be displayed at the appropriate time and at the updated pose. Any number of virtual object(s) VO, VP can be presented during a surgical procedure. In one example, up to twenty different virtual object(s) VO, VP can display various information at various times throughout the steps of the surgical procedure.

During or after the procedure, the controller(s) can transmit, to the remote server RS, any information from the system 10, such as information recognized from the video stream or any contents that are displayed on the HMD 200. These contents can include a video stream transmitted to the HMD 200, a video stream produced by the HMD 200, any text or graphics detected within the video stream and/or virtual objects VO that were displayed on the HMD 200. Other information can be logged, such as user inputs or behavior, system performance data, data transmission or performance, etc. The information can be transmitted for post-operative data analytic purposes or for improving future uses of the HMD 200. The remote server RS can be a cloud server or any suitable type of remote server. Multiple HMDs in the same facility or from multiple locations can communicate to the remote server RS. The remote server RS can include software for analyzing the information from the multiple HMDs to perform any of the described features. The remote server RS can also communicate software updates, calibration settings, or any other information described herein to any HMD 200.

### 3. Example Method(s) Summarizing Technique(s) Involving the HMD

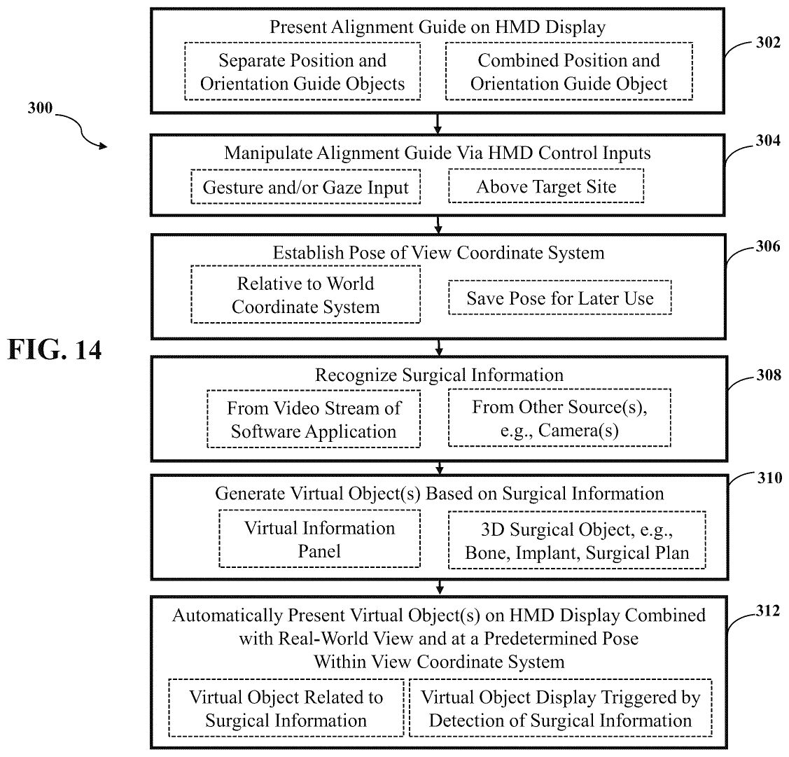

In view of the several techniques described above, FIG. 14 is a flowchart of an example method 300 that may be performed to configure and operate the system 10, connectivity system CS, HMD 200, and/or extended reality system. The steps shown in FIG. 14 are provided for illustrative purposes to explain one example way of operating the system. The steps shown in FIG. 14 are not intended to limit the scope of any technique described herein. Any of the techniques described above can stand alone and provide advantages independently of the other techniques. More of less steps could be part of the method 300 in FIG. 14. Additionally, steps could be implemented in an order different from what is shown and described. The technical details supporting each step have been presented above and are not repeated in detail for simplicity. The steps can be executed by any appropriate controllers/computing systems can include but are not limited to, the camera controller 42, the navigation controller 26, the connectivity system CS, the computing system C, manipulator controller 54, tool controller, or the HMD controller 210. Whatever the applicable system used to perform the method 300 is referred to in this section as the “controller(s)”.

At step 302, the controller(s) can present the alignment guide AG on the HMD display 208. The alignment guide AG can be presented in any manner described, such as including separate position and orientation guide objects PGO, OGO, as shown in FIGS. 6-8. Alternatively, the alignment guide AG may have a simpler configuration, as shown in FIGS. 5B and 5C, for example.

At step 304, the controller(s) facilitate user manipulation of the alignment guide AG on the HMD display 208. This function can be done using gesture and/or gaze input, as described above, for example. The user can also set the position of the alignment guide AG to be directly above the target site TS, for example, as shown in FIG. 7, and can set the orientation of the alignment guide AG to be facing the user's eyes.

At step 306, pursuant to user interaction with the alignment guide AG, the pose of the view coordinate system VCS is established. The VCS can be defined and/or fixed relative to the world coordinate system VCS (e.g., as shown in FIG. 4B.) The controller(s) can save, in a non-transitory memory, the pose of the view coordinate system VCS established by the user. The controller(s) can retrieve the established pose of the view coordinate system VCS during any subsequent use of the HMD by the user. This way, the user/surgeon need not re-calibrate the view coordinate system VCS before every procedure.

At step 308, the controller(s) recognize surgical information. The surgical information can be recognized from numerous sources and in numerous ways, as described above. For example, the controller(s) can obtain surgical information by analyzing text, graphics, and/or specific regions with the video stream of the software application of the host system/device. Additionally, or alternatively, other camera sources can detect the surgical information, such as the camera 214 of the HMD 200 or other HMDs 200 in the operating room, a camera source attached to the tool 22, a camera of the navigation system 20, a camera attached to the manipulator 12, and the like.

At step 310, the controller(s) generate, process, configure, and/or otherwise prepare one or more virtual objects VO based on the recognized surgical information. The surgical information can inform the controller(s) of the type of virtual object VO, the context of the virtual object VO and/or the timing of when the virtual object VO should be presented. The surgical information can be processed in any form, including text, graphics, imagery, and/or video stream. Processing the surgical information can involve any of the techniques described above, including manipulating the surgical information by cropping, clipping, segmenting, replicating, transforming, rearranging, repositioning, and the like. The type, size and pose of any virtual object VO can be generated at this step, and such parameters can be retrieved based on user/default preferences and/or the detected surgical information or step of the procedure. The pose of the virtual object VO relative to the VCS can also be obtained at this step.

At step 312, the controller(s) automatically present one or more virtual object(s) on HMD display 208 combined with the real-world view and at a predetermined pose within view coordinate system VCS. For example, surgical information from the bone registration screen SCR (of FIG. 10) is detected, the controller(s) can automatically present the virtual objects VO at their specific poses within the VCS, as shown in FIG. 11. Subsequently, in the same procedure, when surgical information from the bone preparation screen SCR (of FIG. 9) is detected, the controller(s) can automatically present virtual objects VO related to bone preparation at their specific poses within the VCS.

### C. Improvements in Reducing Latency in Wireless Transmission of Video Data to HMD

As described above, the connectivity system CS is configured to establish connectivity between the HMD 200 and a separate host system/device 20 (such as the navigation system) that is configured to present a software/clinical application on a display.

Described herein are techniques for improving speed of communication or reducing latency in wireless transmission of video data to the HMD 200. These techniques can be used in conjunction with any of the various features or functions of the system 10 described above. For simplicity, the techniques are described as being performed by the connectivity system CS. However, as described above, the connectivity system CS can be integrated, in part, or in whole, into the host system/device or navigation system 20. For example, the connectivity system CS can be implemented by the navigation controller 26 and the components of the connectivity system CS can be incorporated into the cart assembly 24. Also, the connectivity system CS can be integrated, in part, or in whole, into the HMD 200. The techniques described can be performed by any one or more of these described systems/components.

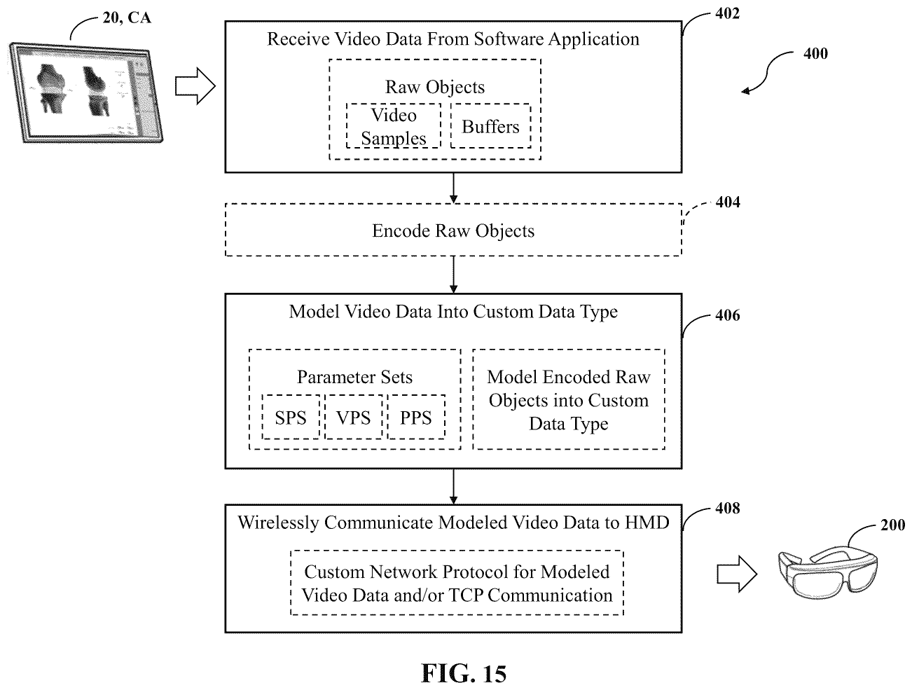

Referring to FIG. 15, an example method 400 is described for processing video data and communicating the video data in a manner that provides significant improvements in reducing latency.

At step 402, the connectivity system CS receives the video data or video stream of the software application or clinical application CA. In one example, the video data is transmitted using wired communication. At this step, the connectivity system CS can use an object that is configured to find capture devices that match specific search criteria to detect the host system/device 20. Once detected, the host system/device 20 can be added as a video input to the connectivity system CS. The connectivity system CS employs an algorithm to receive sample buffers from the video data and to monitor the status of the video data. The connectivity system CS can receive the video data as raw objects containing samples of the video data and a buffer of the video data. The video data can include a plurality of video frames.

At step 404, the connectivity system CS can optionally encode the video data. When raw objects are utilized, the connectivity system CS can encode the raw objects. Encoding the video data maintains the quality of the video data while enabling compression of the video data to reduce latency in wireless transmission to the HMD 200. In one example, the connectivity system CS can use high efficiency video coding (HEVC) such as H265 encoding to compress the video data or raw objects. The connectivity system CS can also employ hardware accelerated encoders and decoders.

The connectivity system CS may utilize transmission control protocol (TCP) communication with the HMD 200. When the connectivity system CS is active, a TCP server listens for incoming network requests at a specified port number. When the HMD 200 is ready to receive video data from the connectivity system CS, the HMD controller 210 sends a network request using the IP address of the connectivity system CS and the specified port number. This implements a TCP handshake. Once the TCP handshake is complete, the connectivity system CS and the HMD 200 are successfully connected to each other, and the connectivity system CS can begin sending video data over the network.

At step 406, the connectivity system CS is configured to model the video data into a custom data type. The custom data type can utilize parameter sets for the video data, or compressed video data. The parameter sets can include Sequence Parameter Sets (SPS), Video Parameter Sets (VPS), and Picture Parameter Sets (PPS). The SPS contains information that is constant throughout the video sequence and information about video resolution, frame rate, bit depth, and other sequence-level settings. The PPS contains information about each video frame. The VPS contains information related to video scalability, which enables the video data to be encoded at different resolutions or quality levels. In cases where the raw objects are encoded, the connectivity system CS can model the encoded raw objects into the custom data type. In other words, the custom data type can take in a compressed object that models a buffer of the video data, e.g., after the object has been encoded. The connectivity system CS can model each frame of the video data into the custom data type. One video frame modeled into the custom data type can include the three parameter sets (SPS, VPS, PPS) and the compressed buffer data.

At step 408, the connectivity system CS, is configured to wirelessly communicate the modeled video data to the HMD 200. The connectivity system CS can utilize a customizable network protocol to facilitate wireless communication of the video data to the HMD 200. The network protocol can be customized specifically to facilitate wireless communication of the modeled video data to the HMD 200. The network protocol can also be customized for communication of the modeled video data to the HMD 200 using the TCP connection. The modeled video data sent over TCP connection as a byte stream. The modeled video data can be represented as a message that is sent over the TCP connection. To support sending the modeled video data over the TCP connection using a byte stream, the customized network protocol is defined and added on top of existing protocol stacks for both the client and the server. One example of the customizable network protocol is one that defines application message parsers.

Using the custom network protocol to define the message alleviates the need to send multiple byte streams over the network for a single video data stream. Using the custom network protocol also alleviates the need to process many byte streams on the HMD 200, e.g., to reconstruct the objects that model the buffer of the video data. In turn, by realizing these advantages, implementation of the customized network protocol decreases latency in wireless communication to the HMD 200.

When the output device OD of the connectivity system CS is a WiFi router, the computing device C of connectivity system CS can be coupled to the WiFi router using a wired connection, such as an ethernet cable. Having a wired connection between the connectivity system CS and the router increases efficiency of data transfer, decreases overall latency, as avoids/prevents packet loss with wireless signal interference. The techniques described herein provide robust and fast processing and communicating of the video stream to the HMD 200, which provides particular advantages for surgical applications.

Several implementations have been discussed in the foregoing description. However, the implementations discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

What is claimed is:

1. An extended reality system for use in a surgical procedure, comprising:
a head-mounted device (HMD) comprising an HMD display positionable in front of a user's eyes and a sensing system configured to sense control inputs of the user; and
one or more controllers coupled to the HMD and being configured to:
receive control inputs from the sensing system to establish a pose of a view coordinate system in which to present a virtual object related to the surgical procedure;
define the view coordinate system relative to a world coordinate system after the pose of the view coordinate system is established;
recognize surgical information; and
in response to recognition of the surgical information, automatically present the virtual object on the HMD display combined with a real-world view and at a predetermined position and orientation within the view coordinate system.

2. The extended reality system of claim 1, wherein the surgical procedure involves a target site, and wherein the one or more controllers are configured to:
receive control inputs from the sensing system to establish a position of the view coordinate system to be located directly above the target site.

3. The extended reality system of claim 1, wherein to establish the pose of the view coordinate system, the one or more controllers are configured to:
present, on the HMD display, an alignment guide configured to assist the user to define the pose of the view coordinate system, wherein the alignment guide is computer-generated and combined with the real-world view on the HMD display.

4. The extended reality system of claim 3, wherein the alignment guide comprises a position guide object dedicated to establishing a position for the view coordinate system and an orientation guide object dedicated to establishing an orientation for the view coordinate system, and wherein the one or more controllers are configured to:
receive control inputs from the sensing system to enable selection and translational movement of the position guide object to establish the position of the view coordinate system; and
receive control inputs from the sensing system to enable selection and rotational movement of the orientation guide object to establish the orientation of the view coordinate system.

5. The extended reality system of claim 4, wherein:
the position guide object is a first volumetric object;
the orientation guide object is a second volumetric object; and
a straight object is virtually coupled between the position guide object and the orientation guide object, wherein the straight object has a fixed length such that the position guide object and the orientation guide object are spatially constrained relative to one another by the fixed length of the straight object.

6. The extended reality system of claim 3, wherein the alignment guide is spatially separate and distinct from the virtual object, and wherein the one or more controllers are configured to:
receive control inputs from the sensing system to enable translational movement of the alignment guide to establish a position of the view coordinate system; and
receive control inputs from the sensing system to enable rotational movement of the alignment guide to establish an orientation of the view coordinate system.

7. The extended reality system of claim 1, wherein the virtual object is related to the surgical information.

8. The extended reality system of claim 1, wherein the one or more controllers are configured to:
receive, from a surgical navigation system, a video stream of a clinical application that is presented on a display of the surgical navigation system;
recognize the surgical information from the video stream of the clinical application; and
in response to recognition of the surgical information, automatically present the virtual object on the HMD display.

9. The extended reality system of claim 8, wherein the one or more controllers recognize the surgical information from the video stream of the clinical application by being configured to automatically identify text and/or imagery presented by the clinical application.

10. The extended reality system of claim 9, wherein the clinical application comprises a plurality of different screens related to the surgical procedure, wherein each screen comprises an identification, and wherein the one or more controllers:
recognize the surgical information from the video stream of the clinical application by being configured to automatically identify the text of the identification of one of the screens of the clinical application.

11. The extended reality system of claim 8, wherein:
the surgical information comprises a step of the surgical procedure; and
the step of the surgical procedure comprises one of: a pre-operative planning step, an operating room setup step, an anatomical registration step, an intra-operative planning step, an anatomical preparation step, or a post-operative evaluation step.

12. The extended reality system of claim 8, wherein the virtual object comprises a virtual information panel that is configured to display information related to the surgical information.

13. The extended reality system of claim 12, wherein the one or more controllers are configured to duplicate a portion of the video stream of the clinical application on the virtual information panel.

14. The extended reality system of claim 13, wherein the one or more controllers are configured to duplicate a navigation guidance region on the virtual information panel, the navigation guidance region displaying one or more surgical objects tracked by a localizer of a surgical navigation system.

15. The extended reality system of claim 1, wherein the virtual object comprises a 3D surgical object including one or more of: a 3D model of a bone, a 3D model of an implant, and a 3D surgical plan.

16. The extended reality system of claim 1, wherein:
the HMD comprises a camera configured to produce a live video stream of the real-world view; and
the one or more controllers combine the virtual object with the real-world view by combining the virtual object into the live video stream.

17. The extended reality system of claim 16, wherein the one or more controllers recognize the surgical information from the camera of the HMD.

18. The extended reality system of claim 1, further comprising a surgical device coupled to the one or more controllers and comprising a camera source, and wherein the one or more controllers recognize the surgical information from the camera source of the surgical device.

19. A head-mounted device (HMD) for use in a surgical procedure, the HMD comprising:
an HMD display positionable in front of a user's eyes;
a sensing system configured to sense control inputs of the user; and
one or more controllers coupled to the HMD display and the sensing system and being configured to:
receive control inputs from the sensing system to establish a pose of a view coordinate system in which to present a virtual object related to the surgical procedure;
define the view coordinate system relative to a world coordinate system after the pose of the view coordinate system is established;
recognize surgical information; and
in response to recognition of the surgical information, automatically present the virtual object on the HMD display combined with a real-world view and at a predetermined position and orientation within the view coordinate system.

20. A computer-implemented method of operating an extended reality system for use in a surgical procedure, the extended reality system including a head-mounted device (HMD) with an HMD display positionable in front of a user's eyes and a sensing system configured to sense control inputs of the user, and one or more controllers coupled to the HMD, the computer-implemented method comprising:
receiving control inputs from the sensing system for establishing a pose of a view coordinate system in which to present a virtual object related to the surgical procedure;
defining the view coordinate system relative to a world coordinate system after the pose of the view coordinate system is established;
recognizing surgical information; and
in response to recognizing the surgical information, automatically presenting the virtual object on the HMD display combined with a real-world view and at a predetermined position and orientation within the view coordinate system.
